(19) United States
(12) Patent Application Publication
(10) Pub. No.: US 2018/0356373 A1
(43) Pub. Date: Dec. 13, 2018

(54) Metastasis and Adaptive Resistance Inhibiting Immunotherapy Combined Online Chemotherapy with Radiotherapy's tumor Seeking Extracellular Vesicles with siRNA and Chemotherapeutics

(71) Applicant: Velayudhan Sahadevan, Beckley, WV (US)

(72) Inventor: Velayudhan Sahadevan, Beckley, WV (US)

(21) Appl. No.: 15/621,973

(22) Filed: Jun. 13, 2017

(51) Int. Cl. G01N 30/02; C12N 15/113; C12N 15/63; C12Q 1/68; G01N 30/04

(52) U.S. Cl. CPC G01N 30/02; C12N 15/1138; C12N 15/113; A61K 48/00; C12Q 1/6876; G01N 30/04; C12N 15/63

(57) ABSTRACT

Mutated genome silencing with endogenous RNAi-siRNA and miRNA with near total cellular apheresis with pulse flow apheresis system and EV-exosome-RNA molecular apheresis with sucrose density gradient continuous flow ultracentrifugation combined with array centrifuge for both 50S higher and 50S lower proteomics and genomics apheresis and their fractionated purification with immobilized Tim4-Fc protein Ca2+ magnetic beads affinity chromatography (ACG) and immobilized metal ACG is disclosed. It purifies normal cell derived and tumor cell derived EVs-exosomes, proteomics and subcellular particles. Tumor-specific endogenous siRNA is generated from mutated RNA containing pre-miRNA hairpin through RNA-induced silencing complex (RISC) composed of Dicer, dsRNA binding protein TRBP, and AGO2. Incubating purified RSIC with pre-let-7 hairpin generates siRNA. SiRNA is bonded with T-EVs and T-cells to silence its evasion from tumor immunity. While on radiation therapy or surgery, a patient's blood is continuously processed with above systems. It delivers combined online radiotherapy, and tumor-seeking adoptive extracorporeal chemo-immunotherapy.

Tumor exosome apheresis is described in the pending non-provisional patent application Ser. No. 15/189,200, “Device and Methods for Broadbeam and Microbeam Chemo-Radiosurgery Combined with Tumor Exosome Apheresis”. This continuation-in-part patent application expands the scope of the prior patent application Ser. No. 15/189,200 to include extracorporeal differential apheresis and plasma pheresis of circulating normal and mutated extracellular vesicles (EVs), DNAs, RNAs, microRNAs, nucleosomes and nanosomes.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

This invention discloses therapeutic control of EVs including exosomes released from normal and cancer cells. The differential cellular continuous flow apheresis (DCCFA) and differential cellular pulse flow apheresis (DCPFA) described this invention separates the components of blood cells with attached tumor cell derived mutated RNAs, DNAs and subcellular particles. The differential continuous flow ultracentrifugation (DCFUC) plasma pheresis is combined with size exclusion chromatography (SEC) and elective affinity chromatography with columns selected from the group of immobilized Tim4-Fc protein Ca²⁺ magnetic beads affinity column (ITMAC), immobilized metal affinity chromatographic columns (IMAC), immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. They separates and purifies cell free DNAs and DNA fragments, RNAs including mRNAs, miRNAs and its tRFs derived from normal cell and cancer cells and their mutated counterparts. Cancer cells shed billions of EVs, exosomes and exRNAs. In response to cancer treatments, surgery, chemotherapy and radiation, much more EVs containing apoptotic bodies, microvesicles, exosome with mutated tumor DNA, RNA, exosomes, microsomes, nucleosomes, miRNAs and tumor associated mutated proteins are released into the circulating body fluids. Therapeutic molecular separation based on physical and biological characteristics of the offending mutated DNA/RNA, exosomes, microsomes, nucleosomes, miRNAs and tumor associated proteins eliminates and or minimizes the tumor recurrence and metastasis.

The contemporary just weeks only median survival of patients with highly aggressive non-Hodgkin lymphoma, the 3.5 months median survival for patients with EMT/MET cancer stem cell predominant sarcomatous mesothelioma (1), less than 2 year overall survival of patients with glioblastoma, pancreatic cancer and unresectable esophageal cancer (2) and others could be improved to longer disease free and overall survival and more cure by mutated tumor derived extracellular vesicle (EVs) controlled cancer treatments.

The EVs-exosomes contains both normal cell and tumor cell derived apoptotic bodies, microvesicles (MVs), exosomes, DNA, RNAs, microRNAs and several kind of normal cell and tumor cell specific proteins. The present patient specific treatments for glioblastoma, based on immunohistochemical identification of EGFR, mutated p53, isocitrate dehydrogenase-1 (IDH-1) and murine double protein2 (MDM2) and treatment consisting of surgery, radiation and chemotherapy could offer only 7.56 months symptoms free survival (3). The tyrosine kinase inhibitor (TKI) chemotherapy based on epidermal growth factor receptor (EGFR) amplification could “sadly” improve the survival of patients with advanced lung cancer only for two months (4). It is not the result we want or hoped for. The lessons we learned from the Big Data on past chemo-radiation therapy (5) and from the genomic knowledge summarized in the p53 database of the International Agency for Research on Cancer (IARC) (6), in the database on microRNAs by the microRNA-cancer association (7) and similar molecular biology databases on other human diseases could be incorporated into patient specific cancer and other illness's treatments with control of billions of EVs carrying apoptotic bodies, microvesicles (MVs), exosomes, oncosomes, DNA and DNA fragments and microRNAs (8).

In response to treatments aimed at cancer and other illness, disease specific mutated EVs carrying apoptotic bodies, microsomes, exosomes, oncosomes, DNA and DNA fragments and microRNAs are released into the tumor, tumor microenvironments and into the blood and lymphatic circulation as well as into pleural effusion, ascites, gastric secretion, saliva, seminal fluids and other body fluids. After their homing into host cells, the benign metastasis and malignant metastasis causing mutated EVs promote angiogenesis through VEGF and initiate the early, niche phase of the metastatic process in the EVs recipient cells. It prepares the recipient cells like those in the lymph nodes for the seeding of the benign and malignant metastasis. Platelet EVs and macrophage activated into tumor promoting M2-like macrophages (9) and the fibroblast MVs promote such metastatic process. Metastatic melanoma EVs travels to sentinel lymph nodes through lymphatic flow and accumulate in the lymph nodes. They attract metastatic melanoma cells into sentinel lymph nodes (9). Lymphangioleiomyomatosis (LAM) cause apparent benign metastasis into the lung (10). M2 like macrophage activated MVs mediates sentinel lymph node benign and malignant metastasis. The metastatic EVs from tumor cell reach the sentinel lymph nodes and prepare the pre-seeding niche in the sentinel lymph nodes. It then attracts the malignant cells from the distant tumor. LAM activated M2 like macrophage reaches the niche in the sentinel lymph nodes. Thus the sentinel lymph node is the very first malignant metastatic site for nearly all the cancers (11) and the benign metastatic site for disease like LAM. It demonstrates the central role of EVs in sentinel lymph node metastasis.

EVs containing mRNAs repairs tissues from stress and enhance cell survival. Hence EVs with mRNAs can repair radiation and chemotherapy induced cellular stress. Human mesenchymal stem cell derived EVs are reported to contain 239 mRNAs (4). They enhance cell differentiation, transcription, cell proliferation and immunity. This shows the difficulty to control tumor cell proliferation with one, two or a few chemotherapeutics since they cannot eliminate or silence all such 239 mRNAs in a single stem cell derived EV. It is further complicated by the presence of a variety of subpopulations of EVs with substantially different cargos and increasing similar ones under hypoxia. Mast cell derived EVs with mRNAs under oxidative stress differs from mast cell EVs with mRNAs under normal conditions. Radiation therapy produce oxidative stress as it generates H₃O++OH ions. Hence after radiation, the EV-mRNA differs from the EV-mRNA before radiation. It enhances the tumor cell recovery from radiation. EVs derived adipocytes stimulate lipid synthesis and storage. The miRNAs secreted by tumor cells, inflammatory and immune cells, blood cells, mesenchymal stem cells and adipocytes and EVs circulating in body fluids is protected from RNAse. Intercellular transfer of EV-miRNAs alters the phenotypes of neighboring tissue cells and in distant tissue cells

EVs and Intercellular Communication

The EVs and its contents including microvesicles MVs, the extracellular RNAs (exRNA) and exosomes control the homeostasis of tissues by intercellular communication through messenger RNAs (mRNAs). Through such intercellular communication and exchange of crucial biomolecules between placenta and endometrium is essential for the successful gestation and pregnancy. They communicate between the endometrium and placenta to induce angiogenic proliferation for the transport of nutrients. The EV facilitating communications between the conceptus, placenta and endometrium is essential for the orderly growth of the fetus. The molecular cargos of these extracellular cell to cell communicating systems with specific miRNAs and proteins are taken up by the endothelial cells and the trophectoderm at the time of implantation. They exemplify the normal roles of these extracellular bodies and the orderly growth and maturation of a fetus and its organs (1).

When the orderly function of the extracellular bodies are altered by genomic mutation of the contents of the EV's and the EV-free circulating apoptotic bodies, DNAs, RNAs, microsomes, nucleosomes and nanosomes, many benign diseases and malignant diseases will result. They include cancer and cancer metastasis and the autoimmune disorders. EVs also remove the cellular derbies. In neurodegenerative diseases, EVs removes both misfolded proteins and transfer aggregated proteins and prion like molecules (2). Through intracellular communication and transfer of mutated DNA, messenger RNA, micro RNA, lipids and proteins, the EVs infects the close by and the distant cells. In cancer, they convert the bystander cells and the distant cells into cells with prometastatic niches that ultimately become metastatic invasion (3)

Cellular and Organ Targeting by EVs

The EV's functions and biological properties are well documented in the review article titled “Biological Properties of Extracellular Vesicles and their Physiological Functions” in the International Journal of Extracellular Vesicles which is incorporated herein in its entreaty (4). The EVs bind to their tissue specific targets through tissue specific receptors and adhesion molecules in their membrane. The EV-membrane-receptor-host cell-membrane bound EVs are taken up by the host cells by phagocytosis or macropinocytosis and transfer their cargos into the host cells (4). EVs transport and secrete cytokines. Sialic acid binding immunoglobulin lectins CD169 (Sialoadhesion) specifically binds α2,3-linked Sialic acids on the surface of the EVs. EV uptake by the host cell is also based on C-type lectin interaction. It is suggested that the reticulocyte uptake by the macrophages is dependent on the interaction of β-galactosides on the surface of reticulocyte and galectin-5 (4). Lactadherin (fat globule—epidermal growth factor-8) bound phospatidylserine (PS) binds to apoptotic cells and platelets derived EVs and upon its binding to ARG-Gly-Asp (RGD) and to αvβ3 and αvβ5 integrins they undergo phagocytosis by macrophages (4) and removed from the circulation. The platelets derived EVs binds to endothelial locus-1 (DEL-1) and to αvβ3 integrins on endothelial cells (ECs). It facilitates the platelet derived EV's uptake into ECs. The EVs containing lipolytic phospholipases interacts with membrane G-protein-coupled receptors in target cells as a means for signal transfer. EVs released by the tumor cells lyses the lymphocytes that are destined to kill the tumor cells. It helps the tumor cell survival. EVs transport chemokines and interleukin family of proteins into extracellular space. EVs secret membrane bound tumor necrosis factor (TNF). Tumor cells and platelets derived EVs secrete vascular endothelial growth factor (VEGF). Thymus derived EVs regulate T cells through Transforming Growth Factor (TGF)-β. Heparin sulfate helps the transfer of EV-TGF-β into recipient cells.

Circulating RNAs

Vesicular EV's cargo and non-vesicular circulating genomics include messenger RNA (mRNA), transfer RNA (tRNA), microRNA (miRNA), small interfering RNA (siRNA), long non-coding RNA (lncRNA), piwi-interacting RNA, ribosomal RNA (rRNA) and fragments of RNAs, tRNA, miRNA, siRNA shRNA and vault and Y-RNAs. These RNAs within the EV's and exosomes are protected from RNAse by their binding to Argonaute proteins. The Ago2 bound RNA is not degraded by RNase. They are abundant in plasma, serum, urine, plural effusion, ascites, chylous, and in saliva.

The 30 to 40 nucleotide length 5′ and 3′ tRNAs is generated from mature t-RNAs by the ribonuclease angiogenin (ANG) (12) which is also known as ribonuclease (RNAse) 5. In proliferating cells, ANG accumulates in the nucleus and stimulate the ribosomal RNA (rRNA) transcription (13). Under stress, ANG moves from the nucleus to cytoplasm and splices the tRNA into short tRNA fragments. Short tRNA includes tRNA halves (tiRNA), tRNA derived fragments (tRFs) and miRNA like fragments-the miR-TRFs (13, 14, 15). The tRFs behaving like miRNAs but not being derived from a canonical pri-miRNA gene include tRF CU1276, miR-720 (tRNA^{Thr}), miR-1274b (tRNA^{Lys}), miR-4284, miR-1308, human stem cells hsa-miR-4284, melanoma hsa-miR-3182, hsa-miR-1280, and hsa-miR-886-5p (15).6The miR-tRFs 720 and miR-1274b are secreted into EV-exosome derived from breast cancer cells (16). The point mutation in both the nuclear tRNA (n)(tRNA) and the tRNA moved to cytoplasm, the mitochondrial tRNA (mt)tRNA are associated with various benign diseases and in cancer (17, 18). Some of the diseases associated with (mt)RNA and (n)tRNA include cardiomyopathies, encephalopathies, chronic progressive external ophtalmoplegia, (CPEO), mitochondrial encephalomyopathy lactic acidosis stroke-like episodes (MELAS), mitochondrial myopathy (MM), and myoclonic epilepsy with ragged-red fibers (MERRF) (17). Oxidative stress from nephrotoxic cisplatin and renal ischemia generate tiRNAs (15) which cause renal insufficiency. Few of the (mt)tRNA associated neoplasia includes splenic lymphomas, breast cancer, gastric, hepatocellular, head and neck cancers, multiple myeloma, gynecological tumors, colon carcinoma and nasopharyngeal cancers. Likewise, few of the (n)tRNA associated cancers include multiple myeloma, breast cancer, ovarian cancer, colorectal cancer and osteosarcoma (17). The tRNA fragments from estrogen receptor positive breast cancer and androgen receptor positive prostate cancer generate sex hormone dependent small RNAs (15) that accelerate tumor growth.

Both normal cell and tumor cell derived EVs contain classical miRNAs while the tumor cell derived EVs also contain miRNA like fragments that is not found in the parent tumor cells. These miRNA like-tRNA fragments have gene silencing activity. The EVs derived from MCF7 and MCF10A breast cancer cells (16) are rich in miR-21, let-7a, miR-100, miR-125 and low in miR-205. The miRNA like tRNA fragments (tRFS), miR-720 and miR-1274b were found only in EVs derived from MCF7 and MCF10A breast cancer cells. They may have silencing activity (16). The miRNA like-tRNA fragments binds to Argonaute proteins. Hence they could also silence the gene expression of tRFs (19). Based on the molecular differences in miRNA and tRNA fragments in tumor cell derived EV's but not in normal cell derived EVs, they could be separated and purified by DCCFA, DPFA, DCFUC combined with SEC IEC and AC as cell free DNAs and DNA fragments, RNAs including mRNAs, miRNAs and its tRFs.

Identification of the association of normally occurring and disease associated difference in tRNA is important in molecular diagnosis and cancer treatment. The tRNA fragments, i-tRFs with differing characteristics within races and in disease and in normal tissue are an example of the opportunities it lends for disease specific diagnosis and treatments (20). They have varying nt lengths and constituents. The nuclear tRNAs differs from the mitochondrial tRNAs. There is an abundance of tRNAs within tRNA space; it contains 505,295 tRFs. The racial difference in tRNA is noted among western European, Finnish, British, Toseani Italian and Youraba Africans from Ibadan ancestries. TRFs are tissue specific. Its abundance depends on disease type and subtype. Estrogen receptor (ER), progesterone receptor (PR) and HER2 positive and ER, PR and HER2 negative, triple negative, breast cancers have differentially abundant tRFs. There is a lower abundance of tRFs in the tumors as compared to normal tissue. The identification of the difference in tRNA fragments like i-tRFs in diseases including in triple positive and negative breast cancer is of great significance for molecular diagnosis and treatments. The triple positive tumor tissue is characterized by low abundance of 17 tRFs while the triple negative breast cancer is characterized by low abundance 19 tRFs (20). The tRFs are loaded on to Argonauts proteins. The differential Argo loaded tRFs can identify the tumor tissue from the normal tissue and within the tumor tissue it can differentiate the triple positive tumors from triple negative tumors (20). Circulating cell free RNAs include the mRNAs, miRNAs and its tRFs (21). The tiRNAs guides the Argonaute proteins on to the complementary sites on RNA. The molecular weight of Argonaute protein is about 100 kDa (21). The tRNAs and tiRNAs with differing number of nt itself and the tRNAs and tRFs bound to the Argonaute proteins changes their molecular weights. Protein molecular weight standards like thymoglobulin, bovine serum albumin, ribonuclease and tyrosine include 669 kDa, 67 KDa, 13.7 KDa and tyrosine 0.181 kDa respectively. Based upon such molecular weight differences, they are eluted in distinct elution fractions (22). Likewise, in sucrose density gradient continuous flow ultracentrifugation, the plasma contents with varying molecular weights are fractionated in dissentingly separated fractions. Hence they could be separated by differential density gradient ultracentrifugation and differential chromatography described in this invention. It facilitates the identification of the difference in tRNA fragments like i-tRFs in varying diseases and within diseases like in triple positive and negative breast cancer (20).

Pulse Flow Cellular Apheresis

The pulse flow apheresis combined with affinity chromatography is used to remove the CTCs and most of the apoptotic bodies, microsomes, nucleosomes and exosomes release after chemotherapy, radiosurgery and chemo-radiosurgery but it leaves still present circulating cell debris, cell membranes, and plasma soluble normal tissue derived and tumor tissue derived apoptotic bodies, DNA and RNAs, microsomes, exosomes and nanosomes, telomere and telomerase, ATM and ATM kinase. These tumor derived micro and nanosomes causes bystander effects and abscopal metastasis. They are removed by therapeutic continuous flow ultracentrifugation plasmapheresis.

Continuous Flow Blood Separator

Alternative to pulse flow apheresis, the continuous flow blood separator could be used for apheresis (23) but it has many disadvantages for the separation and removal of circulating cell debris, cell membranes, and plasma soluble normal tissue derived and tumor tissue derived apoptotic bodies, DNA and mutated RNAs, microsomes, EVs-exosomes, nanosomes, telomere and telomerase, ATM and ATM kinase. Its efficiency of cell separation such as the platelet separation ranges from 47.18% to 63.22% (23 C). It is too low for efficient treatment of mutated genes in the circulation where near total separation and elimination of mutated genes is required. Because of the mutated exosome size can be as low as 100 nt, the low speed continuous flow centrifugation cannot separate them from the plasma and those that are cell bound. However, the continuous flow blood separator could be used to separate the white blood cells, platelets and the plasma. It cannot be used for differential separation normal cell derived EVs-exosomes and tumor cell derived mutated EVs-exosomes. They have only a few kDa molecular weight differences. However, it has been used to remove plasma soluble antigen antibody complex and to overcome chemotherapy resistance. Such use was reported in the treatment of a case with neuroblastoma (24). In this case, the pre-plasmapheresis chemotherapy showed no increased VMA but the post-plasmapheresis chemotherapy showed highly increased VMA. It indicates removal of interfering molecules by plasmapheresis sensitizes chemotherapy. Still, since it does not remove the millions of tumor EVs-exosomes and nanosomes, continuous flow bolls cell centrifugation is proven to be ineffective for plasmapheresis combined chemotherapy.

Continuous Flow Ultracentrifugation Molecular Plasmapheresis of Circulating Tumor Derived EVs

The plasma partially purified by pulsed flow plasmapheresis combined with siRNA affinity chromatography is diverted to a continuous flow ultracentrifuge for additional sucrose density gradient (SDG) ultracentrifugation for further removal of the circulating plasma soluble normal cell and tumor cell derived cell debris, cell membranes, and tumor associated proteins, apoptotic bodies, DNA and RNAs, microsomes, EVs-exosomes and nanosomes, telomere and telomerase, ATM and ATM kinase. They could cause metastasis. Mutated telomerase surge after therapeutic intervention could lead to aberrant DNA repair leading to eventual tumor recurrence and metastasis. In this invention, the surge of plasma soluble tumor derived micro particles, EVs-exosomes, telomere and telomerase and damaged DNA/RNA after cancer treatments, high dose radiation/radiosurgery and combined radiosurgery and chemotherapy is removed by therapeutic DCCFA, DPFA, and DCFUC combined with SEC IEC and AC.

The principle of continuous flow ultracentrifugation was pioneered at the Oak Ridge National Laboratory for the U.S Atomic Energy Commission under the Molecular Anatomy Program (The MAN Program) and was cosponsored by the National Cancer Institute (NCI), the National Institute of General Medical Sciences (NIH), the National Institute of Allergy and Infectious Diseases (NIAID) and the U. S. Atomic Energy Commission. It was conducted under the leadership of N. G. Anderson over 50 years ago (25). It is widely used in vaccine preparation against viruses (26) and in micro and nanoparticle cellular research. It is an ideal tool for therapeutic plasmapheresis to remove tumor specific plasma soluble ell debris, cell membranes, tumor derived proteins, apoptotic bodies, DNA, DNA fragments, RNAs, microsomes, EVs-exosomes and nanosomes, telomere and telomerase, ATM and ATM kinase surge after therapeutic intervention. Continuous flow cell centrifuge plasmapheresis without ultracentrifugation is used safely to treat a variety of diseases but they are incapable of removing nanometer sized tumor derived particles (23). Its centrifugation speed is only about 1,500 rpm (27) while the nanoparticles like the size of a virus are removed at 40,000 rpm at 100,000 G (28). Although the continuous flow ultracentrifugation technology has been richly developed for virus research and vaccine preparation (26, 25), it is not yet in use for routine therapeutic clinical applications especially for nanoparticle plasmapheresis as part of cancer treatment. Such treatments decrease the abscopal metastases caused by mutated circulating genes by EVs-exosomes. The principles of continuous flow ultracentrifugation of N. G Anderson (25) were originated from the NCI, NIH and U.S. Atomic Energy Commission half a centaury ago; it is high time for its routine clinical application for more curative cancer treatment. Anderson had the foresight even for its use in immunotherapy of cancer, he and his colleagues showed that an adenovirus membrane could effectively immunize against tumor growth (29).

In this invention, the plasma after pulsed flow combined siRNA-affinity chromatography is diverted to a continuous flow ultracentrifuge for additional SDG ultracentrifugation plasmapheresis of micro and nano particles followed by immune-affinity chromatography that removes the remaining tumor derived plasma soluble cell debris, cell membranes, tumor specific proteins, apoptotic bodies, DNA and RNAs, microsomes, EVs-exosomes and nanosomes, telomere and telomerase, ATM and ATM kinase. On the basis of differential molecular weights and configuration, the normal cell derived EVs-exosomes are separated from tumor cell derived EVs-exosomes. It facilitates their separation and purification by continuous flow ultracentrifugation. However, it is suitable for separation of proteomics and genomics with higher than 50S.

Prior Art Affinity Chromatography with Lectins

U.S. Pat. No. 9,364,601 teaches removal of circulating exosomes by lectins affinity chromatography or by exosome specific antibody bound exosome removal as an adjuvant to cancer treatment (30, 30B). It is incorporated herein in its entirety. In this process, the protein bound RNA is hardly removed. The total RNA content in per milliliter exosome is substantial, however the protein bound circulating RNA is ten times more than the RNA in the exosome (31). The serum derived exosome contains only about one RNA molecules per exosome. However, because of their abundance, about 10¹² exosomes per milliliter body fluids, exosomes are rich in RNAs. Within the limited volume of an exosome, some of their subpopulation is filled with more RNA and fewer proteins while others are filled with more proteins and fewer RNA (31). The extracellular vesicles (EVs) in the body fluids include the apoptotic bodies, microvesicles (MV) and exosomes. Circulating tumor derived EVs are capable of inducing tumor recurrence and metastasis (32). Hence removing only part of the EVs, like the smaller molecule exosome filtration from the circulation is not very effective in cancer treatment. Exosomes are also present in larger cells, in red blood cells, platelets and white blood cells. These cells are not removed by the lectins based exosome filtration methods. The methods of antibody bound exosome remove only those exosome to which specific antibody is present. There are several types of exosomes and their subpopulations present in the circulation. Hundreds of exosome removal by specific antibody binding is not clinically practical. Hence the antibody bound exosome removal taught in U.S. Pat. No. 9,364,601 (30) is an incomplete therapeutic exosome removal. Furthermore, the lectins and the antibody bound exosome filtration as in U.S. Pat. No. 9,364,601 (30) removes both the circulating normal cell derived exosomes and the circulating tumor cell derived exosomes. The normal cell derived exosomes have important physiological functions. For example, exosomes contains miRNAs that have important cardiac regulatory functions. Micro RNA deficiency could lead to cardiac diseases and heart failure, central nervous system diseases, immune system diseases and other illness (33). It can cause serious clinical complications especially if the patient is also treated with chemotherapeutics like Herceptin or Adriamycin. Both Herceptin and Adriamycin are commonly used chemotherapeutics and they have high rate of cardiac toxicity. The differential tumor cell derived exosome removal combined with normal cell derived exosome preservation avoids such serious clinical complications.

EVs and exRNAs Associated Diseases

In diseases, the EV's cargo and the freely circulating apoptotic bodies, very low levels of DNAs, exRNAs, are carriers of many benign and malignant diseases including cancer and cancer metastasis and autoimmune disorders. The ancient group of 20 enzymes, one for each amino acid known as aminoacyl tRNA synthetases (AARSs) catalyzes the basic genetic code reaction, AA+ATP+tRNA to AA-tRNA+AMP+PPᵢ. It legates the amino acids to its specific tRNAs and thus controls the fundamental genetic codes. Many diseases including cancer, neurological, autoimmune and metabolic disorders are connected to their specific mutated aminoacyl tRNA synthetases, the AARSs (34). Mutated AARSs with different quantities and qualities of amino acids are present in Burkett's lymphoma, prostate and breast cancer, colorectal cancer, sarcoma and pituitary adenoma (20, 34, 35). They are present in the EVs and non-EV circulating apoptotic bodies, DNAs, RNAs, microsomes, nucleosomes and nanosomes. Their molecular and molecular weight differences render the separation of circulating EVs and in exEV by DCCFA, DPFA, DCFUC, SEC, IEC and AC as described in this invention.

EVs and ex RNA guided diagnosis and treatments are applicable to a wide range of diseases. Among the neurological abnormal RNA metabolism associated diseases include cancer (7), childhood-onset spinal muscular atrophy, pontocerebellar hypoplasia and myelination deficiencies (5) and others including diffuse intrinsic pontine glioma (DIPG) (5B). The RNPs in the cell is formed by binding of protein-coding and non-coding RNAs and the RNA binding proteins. The RNPs normal function depends on the integrity of both RNA and its binding proteins. There are a vast number of RNAs and RNA binding proteins. Mutation in RNAs or in RNA binding proteins associated diseases includes the pre-mRNA splicing cis and trans mutation associated diseases (7). Alternative splicing of CD 44 prepares the rat pancreatic adenocarcinoma cell lines for metastasis. CD 44 mRNA isoforms lacking variable regions (CD44 variable or CD44v) is present during development and T-cell activation. CD44v5, CD44v6 and CD44v7 are found in cancer cells. CD44v6 is present on the surface of a variety of metastatic cell lines but not on non-metastatic cells (7).

The alternative cis mutation associated diseases include familial isolated growth hormone deficiency type II (IGHD II), Frazier syndrome associated with inactivation of Wilms tumor suppressor gene (WT1), mutations in the MAPT gene that encodes tau which cause frontotemporal dementia and Parkinsonism, Pick's disease and frontotemporal dementia by aggregation of the microtubule-associated protein tau, atypical cystic fibrosis caused by loss of function of the cystic fibrosis transmembrane conductance regulator (CFTR) gene that encodes a cAMP dependent transmembrane chloride channel (7). It is expressed in secretory epithelium. The other RNA splicing trans mutations associated diseases include retinitis pigmentosa presenting as progressive retinal degeneration, night blindness, loss of peripheral vision, that ultimately leads to total blindness. It is due to loss of rod photoreceptor cells caused by autosomal dominant RP genes, PRPF31, HPRP3, and PRPC8 mutations and spinal muscular atrophy and myotonic dystrophy (7). The conventional treatments combined extracorporeal EVs and the causative genomics' removal from the circulation by the methods of differential cellular and plasma pulse apheresis combined with continuous flow ultracentrifugation molecular apheresis and size exclusion chromatography and affinity column chromatography disclosed in this invention improves the disease control and cure and patient's survival.

Lymph Node Metastasis and Circulating EVs and exRNAs

Platelet EVs and macrophage activated into tumor promoting M2-like macrophages (9) and the fibroblast MVs promote metastatic process. Metastatic melanoma EVs travels to sentinel lymph nodes through lymphatic flow and accumulate in the lymph nodes. They attract metastatic melanoma cells into sentinel lymph nodes (9). Lymphangioleiomyomatosis (LAM) cause apparent benign metastasis into the lung (10). M2 like macrophage activated MVs mediates sentinel lymph node benign and malignant metastasis. The metastatic EVs from tumor cell itself reach the sentinel lymph nodes first and prepare the pre-seeding niche in the sentinel lymph nodes and then attract the malignant cells from the distant tumor. LAM activated M2 like macrophage reaches the niche in the sentinel lymph nodes. Thus the sentinel lymph node is the very first site for metastasis for nearly all cancers (11) and for benign metastasis for disease like LAM. It demonstrates the central role of EVs in sentinel lymph node metastasis.

Metastatic seedlings from peritoneal fluid spread to mediastinal lymph nodes through thoracic duct before its systemic dissemination and metastasis (11B). EVs with mutated genomics systemic spread is often through peritoneal-thoracic duct route to mediastinum and blood flow. Likewise, EVs with mutated genomics spread is through sentinel lymph node-thoracic duct-blood stream route. They lead to systemic metastasis. Cancer treatments especially the focal radiation therapy releases more EVs with mutated genomics spontaneously. Hence, cancer treatments combined with early extracorporeal EV-molecular apheresis minimize or eliminates metastatic cancer dissemination.

Metastatic rat adenocarcinoma BSp73ASML (ASML) prepares the draining lymph nodes and the lung tissue for premetastatic niche formation by CD44v4-v7 knockdown (CD44v^{kd}). Subcutaneous injection of ASML-CD44v_{kd} exosomes containing messenger RNA (mRNA) and microRNA (miRNA) is preferentially taken up by lymph node stroma and lung fibroblasts (34). The early detection of the common primary lymph node metastasis causing circulating CD44, mRNA and microRNAs will lead to metastasis controlled cancer treatment and much improved disease free and overall survival of patients with cancer and cancer cure. Tumor cell derived exosomes enrich the mesenchymal stromal microenvironment. It prepares for the bone metastasis. Extracorporeal EV-apheresis of circulating CD44, mRNA and microRNAs and tumor cell derived EVs homing at mesenchymal stromal cell microenvironment described in this invention opens new avenues for more effective treatments to inhibit lymph node and bone metastasis.

Large Oncosome and Metastasis

Methods of molecular diagnosis and treatment of prostate cancer metastasis based on its large oncosomes of the sizes ranging from 0.1 to 1 micrometer and higher and smaller exosomes of 50 to 100 nm is taught in US patent application 2016/0061842. It is incorporated herein in its entirety (35). It teaches early diagnosis and treatment of prostate metastasis on the basis of large oncosomes. After determining the likelihood of cancer metastasis on the basis of presence or absence of large oncosomes, patients are treated by surgery, radiation therapy, chemotherapy, immunotherapy vaccination or by their combinations. If increased levels of cancer metastasis causing Ck18, Akt, miR 1227, PTEN or their combinations are present, patients are treated by conventional methods of cancer treatments. The theoretical reasoning for such early molecular image guided treatments for metastasis is based on the kinase activity in large exosomes induces angiogenesis. Together with c-Myc, Akt kinases activate cell proliferation and metastasis. Kinase inhibitors minimize this cell proliferation and metastasis. It is an excellent start; however, it is not possible to inhibit all the examples of cancer genes listed in Table 1 in this patent application (35) with clinically safe doses of kinase inhibitors alone or in combination with other chemotherapeutics or by radiation therapy. In addition to large oncosomes shed by cancer cells, tumor cells also shed billions of EVs that also contains apoptotic bodies, micro vesicles (MVs) and exosomes with mutated genomes. The mutated gens in these EVs also cause metastasis. They also need to be treated or eliminated as described in this invention.

Extracellular RNA (exRNA)

The extracellular RNA (exRNA) also known as exosomal RNA are the RNAs present outside the cells from which they are derived. The exRNA are associated with EVs, ribonucleoprotein complexes and lipoprotein complexes. This group of RNAs includes the RNA in the EVs and in exosomes and the RNAs tightly bound to proteins and to lipids (36). They include the messenger RNA (mRNA), transfer RNA (tRNA), microRNA (miRNA), small interfering RNA (siRNA) and the long non-coding RNA (lncRNA). The RNAs inside the cell, the ribosomal RNAs (rRNA) is usually not found outside the cell. The small RNAs include the microRNA (miRNA), piwi-interfering RNA (piRNA), small interfering RNA (siRNA), small nuclear RNA (snoRNA), tRNA-derived small RNA (tsRNA), small rDNA-derived RNA (srRNA) and small nuclear RNA, also known as U-RNA (37). The plasma exRNA consists of 40% miRNA and 40% piwiRNAs while the other known plasma exRNAs like the long non-coding RNAs (lncRNAs), tRNAs and mRNAs consists of only about 2% (38). The non-coding RNA interference (RNAi) consists of endogenously derived miRNA or exogenously derived siRNA (39). They bind to their specific mRNA and either increase or decrease the mRNA's activity in protein synthesis. Dicer enzyme cleaves the long double stranded RNA (dsRNA) into short double stranded siRNAs. Each siRNA unwound into two single stranded RNAs (ssRNAs). One of these two ssRNA functions as a passenger strand and the other one as a guide strand. The passenger strand ssRNA is degraded and the guide ssRNA strand is incorporated into RNA induced silencing complex (RISC) which also contains the catalytic Argonaute 2 protein, (Ago2). When the dsRNA is an exogenous origin like the viral RNA, it directly binds to cytoplasm and the Dicer enzyme cleaves it into short RNAs. If the dsRNA is the endogenous pre-microRNAs, it is processed to form the stem-loop structure of the pre-miRNAs in the nucleus and then exports it into cytoplasm where they bind to Ago2 containing RISC. Exogenous dsRNA that comes from, a virus RNA or is a synthetic RNA binds directly to RISC. Endogenous dsRNA that is first processed to stem-loop structure of pre-miRNA in the nucleus and then exports it into cytoplasmic RSIC containing Ago2. The dsRNA is cleaved into short RNAs (siRNAs) by the Dicer enzyme. The siRNAs base paired with the target specific mRNA silences the mRNA by its cleaving. The miRNA bound RISC on the other hand scans the cytoplasmic mRNAs for complementarity and binds to 3′ untranslated region (3′UTR) of the mRNAs and binds to imperfect complementarity and blocks the access to ribosomes for translation. Thus, it is different from the RISC-Ago2-siRNA mediated mRNAs cleavage. Structurally, miRNA is similar to siRNA but it is produced from the 70 nt long stem-loop structure called pre-miRNA. The pre-miRNA is processed in the cell nucleus by the microprocessor complex consisting of RNase III enzyme called Drosha and a dsRNA binding protein called DGCR8. The dsRNA portion of this pre-miRNA is cleaved by the Dicer enzyme to produce mature miRNA. Like the siRNA, the miRNA is also integrated into the same RISC-Ago2 complex. Argonaute proteins are localized in the cytoplasmic P-bodies which are also called GW bodies. There is a high rate of mRNA decay and high rate of miRNA activity in the cytoplasmic P-bodies (GW bodies). The RNA induced transcriptional silencing (RITS) is carried out by the RITS protein complex. The 3′UTRS of the mRNAs contain binding sites for miRNAs and for regulatory proteins. The epigenetically altered miRNAs impairs the DNA damage repair which in the long term leads to DNA mutation and cancer recurrences (40) Disregulation of gene expression is also associated with neuropsychiatric disorders (39). Among the small RNAs, the miRNA is the best characterized. The EVs and exosomes transport miRNAs. Mi RNAs in the EVs and in exosomes and the miRNAs tightly bound to Argonaute proteins or lipoprotein particles are not destroyed by RNAse. These basic characteristics and molecular weight differences of the various RNAs in circulating extracellular vesicles (EVs) and exosomes and of the extracellular RNAs (exRNAs) makes their separation feasible and removal from the circulation by therapeutic DCCFA, DPFA, and DCFUC combined with SEC IEC and AC described in this invention.

Cancer Stem Cell EVs and exRNAs and Cancer Treatments

Radiation therapy, chemotherapy and surgery removes most of the differentiated cancer cells but fail to cure or control a large number of cancers due to their inability to eliminate CSCs.

A tumor mass contains a small proportion of cancer stem cells that are resistant to most treatments. After varying periods of dormancy, these residual cancer stem cells undergo differentiation and proliferation to become recurrent and metastatic tumors. Hence, most often, there is no lasting tumor control and cancer cure. Representative cancer stem cell surface markers include CD34+/CD38 in AML, CD44+/CD24−/low ALDH in breast cancer stem cells, CD133 in glioma cancer stem cell, CD133 CD44/EpCAM/CD166 in colon cancer stem cell, CD133+/CD26+ in metastatic colon cancer stem cell, CD20, CD271 in melanoma cancer stem cell, ESA/CD44/CD24 in pancreatic cancer stem cell, CD133/CXCR4 in metastatic pancreatic cancer stem cell, CD44/a2β1/CD133 in prostate cancer stem cells, CD133 in lung cancer stem cell, EpCAM/AFP in hepatoma cancer stem cells, CD44 in gastric cancer stem cells (41). The methods of fluorescent antibodies, flow cytometry and cell sorting techniques identify these cancer stem cell markers in these tumors. The cell cycle, apoptosis, EMT/MET, proliferation, invasion and metastasis by these cancer stem cells are controlled by miRNAs and lncRNAs. The miR-22 targets TET2 gene in leukemia and myelodysplastic syndrome CSC. Let-7 targeting RAS and HMGA2, miR-200 family targeting ZEB1/ZEB2, BMI-1 SUZ12 and miR-22 targeting TET1-3, TET family controls the cellular process in CSC in breast cancer. The mir-9/9*, mir-17 targeting CAMTA1 gene, miR-128 targeting BMI-1 gene, and miR-199b-5p targeting HEST gene are known to control the cellular process in CSC in brain tumor. MiR-193 targeting PLAU gene and K-RAS gene, miR-451 targeting MIF and COX-2 genes and miR-34a targeting NOTCH 1 controls the cellular process in CSC in colon cancer. MiR-34a targeting CD44 gene and miR-320 13-catenin gene controls the cellular process in CSC in prostate cancer (41).

The less toxic dietary derived CSC targeting newer drugs like the salinomycin and resveratrol and the antipsychological drug thioridazine are capable of eliminating CSC (41) However, they are incapable of total ablation of the cancer. While the CSC based therapy resistant, triple negative metastatic breast cancer regression is excellent on salinomycin treatment, it is still a partial regression (42). Due to adaptive resistance, to salinomycin, the salinomycin responsive mesenchymal breast cancer cells transformed into salinomycin resistant E-cadherin and miR-200c positive epithelial cancer stem cells (43). It explains the partial response to salinomycin by the triple negative CSC breast cancer that was non-responsive to conventional chemotherapy (42). Since the cancer stem cell derived EVs and exosomes with exRNAs are molecularly different than the normal cell derived EVs and exosomes, they are amiable for removal from circulation. Hence, systemic and extracorporeal cancer stem cell chemotherapy with such compounds combined with endogenous siRNA gene silencing described in this invention and DCCFA, DPFA and DCFUC plasmapheresis of cancer stem cell derived EVs and exosome inhibits cancer stem cell proliferation based tumor recurrence and metastasis.

MicroRNAs (miRNAs)

More than 50% of microRNA is found in human cancers. By upregulation of downregulation, the miRNAs control cell proliferation and apoptosis (44). Mir-let-7a and miR-let 7b are p53 dependent tumor suppressors. Mir-let-7 is up or down regulated upon radiation. P53 dependent mir-34 and mir-21 upregulation or downregulation also controls cell proliferation apoptosis (45). Some of the miRNAs affecting radiation resistant and very poor prognostic cancers are listed below.

Global suppression of miRNA is achieved by downregulation of AGO2 or DICER proteins with RNAi without affecting the DNA damage repair (46, 47). Reduction in AGO2 or DICER leads to increased cell death after radiation indicating prosurvival function of miRNA (31). In this invention, complete or nearly complete removal of circulating Ago2- and DICER is achieved by extracorporeal continuous flow molecular ultracentrifugation plasmapheresis. Cancer treatments like radiation therapy alone or combined with chemotherapy followed by removal of circulating AGO2 and DICER containing EVs and gene silencing with microRNA or siRNA results in increased cancer cell death, decreased tumor recurrence and metastasis or their complete elimination. Such treatment improves the prognosis and survival of patients with very poor prognostic miRNA associated cancers. The commonly occurring miRNAs in poor prognostic EMT/MET sarcomatous mesothelioma, glioma and glioblastoma multiforme, pancreatic cancer, locally advanced carcinoma of the esophagus, HPV negative head and neck tumors, locally advanced non-small cell and small cell lung cancers and triple negative breast cancer are listed in the table below.

TABLE 1
Poor Prognosis Cancer Associated miRNAs

| No | Somatic Mutation | miRNAs |
|---|---|---|
| 1 | CDKN2A/alternative | miR-205, miR-31, miR-17-5p, miR-21, miR-29a, miR-30c, - |
| SM | reading frame | miR-30-e-5p, miR-106a, mir-143, miR-mir-141, miR-192, |
| | (ARF), CDKN2B | miR-200a-c, miR-203, miR-429, miR135b, miR-181a-2, |
| | NF2, | miR-499-5p, miR-519d, miR615-5p, miR-624, miR-218-2, |
| | BRCA-associated | miR-314, miR-377, miR-485-5p, miR, miR-525-3p, miR- |
| | protein (BAP1)- in | 301, miR-18, miR-433, miR-543-3p, miR-584, miR-885-3p, |
| | tumor cell; | miR-7-1, miR-9, mIR-30b, miR-32, miR-483-3p, miR-584, |
| | Ref. 22), | miR-429, miR-17-5p, miR-20a, miR-92, miR-145, miR-1, |
| | MGMT, RASSFIA, | miR-16, miR-29c, miR-31, miR34b, miR-34c, miR-193a- |
| | DAPK, RAR, | 3p, miR-21-5p, miR-23a-3p, miR-222-3p. |
| | RASSFIA, DAPK, | (Ref. 24, 25, 26) |
| | RARβ, p16(INK4)- | |
| | in pleural fluid | |
| | and in tumor | |
| | (Ref. 23 ) | |
| | CDKN2A | |
| | WT1, NF2, TP53 | |
| | BAP1, PTEN, | |
| | MSLN | |
| | MME, LATS2, | |
| | THBS1 | |
| | DTX2P1-UPK3B | |
| | (Ref. 27) | |
| 2 | MGMT, IDH1, | miR-7, miR-124, miR-137, miR-21, miR-34a, miR- 128, |
| GM | LOH, EGFR, p16, | miR-196b, miR-125a, miR-29b, mir-146b-5p, miR-124a, |
| | DAPK, RASSFIA, | miR-101, miR-31, miR-195, miR-137, miR-125b, miR-483- |
| | p73, RARbeta, | 5p, miR-196b, miR-145, miR-128, miR-326, miR-10b, miR- |
| | PTEN, p15INK4B, | 143, miR-153, miR-129, miR-136, miR-342, miR-376, miR- |
| | p14ARF | 219-5p, miR-92b, mir-223, miR-106a, miR-331-3p, miR- |
| | (Ref. 20) | 100, miR-503, miR-101, miR-222, miR-31, miR-330, miR- |
| | PTEN, MMP2 | 148, miR-196a, miR-127-3p, miR-18a, let7g-5p, miR-320, |
| | MGMT, KRIT1 | miR-320, miR-340, miR-576-5p, miR-626, miR-7-5p, miR- |
| | PDGFA, DMBT1 | 142-3p, miR-149, miR-320a, miR-320a, miR-210-3p, miR- |
| | MIR21, NOTCH2 | 152, miR-449, miR-210, miR 3189-3p, miR-873, mi 212-3p, |
| | GFAP, RTEL1 | miR-577, miR-139-5p, miR-135-b, miR-137, miR-663miR- |
| | CCM2, OLIG2 | 25, miR-130b, miR-190b, miR-181b, miR-542-3p, miR-454, |
| | PDCD10, CIC | miR-429miR-517c, miR-590-3p, miR-622, miR-181c, miR- |
| | S100A6, RAF1 | 506, miR-133b, miR183, miR-138, miR-296-5p, miR-519a, |
| | ROS1, TGFB2 | miR-181c, miR-154, miR-197, miR-143, miR-595, miR- |
| | LRP5, NKX2-2 | 300, miR-369-3p, miR-323a-3-p, (Ref. 7) |
| | FAT1, IQGAP1 | |
| | PTPRK, CCDC26 | |
| | GLTSCR2 | |
| | (Ref. 28) | |
| 3 | BRAF, CTNNB1, | miR-21, miR-34a, miR-34b, miR34-c, miR-146a, miR-96, |
| PC | GNAS, KRAS, | miR-17-5p, miR-15a, miR-214, miR-132, miR-212, miR- |
| | PIK3CA | 10a, miR92, miR-301a, miR-148b, miR-216a, miR- 124, |
| | (Ref. 19) | miR-375, miR-424-5p, miR-150, miR-630, miR-148a, miR- |
| | TP53 | 198, miR141, miR-221, miR-150-5p, miR-152, miR-99a, |
| | (Ref. 6) | miR-27a, miR-196a, miR-203, miR-181a, miR-1247, miR- |
| | MUC1 | 497, miR-373, miR-335, miR-191, miR-133a, miR-216, |
| | CEACAM5 | miR-7, miR-1181, miR-145, miR-410, miR-615-5p, miR- |
| | MUC6, ACHE | 29c, miR-367, miR-221, miR-222, miR-101, miR-301a-3p, |
| | SSTR2, CXCR4 | miR-33a, miR-15b, miR-29a, miR-744, miR-181c, miR- |
| | CCK, MUC4 | 150, miR-215, miR-494, miR-329, miR-1271, miR-506, |
| | SMAD2, GLI1 | miR-320, miR-145. (Ref. 7) |
| | PRSS1, STK11 | |
| | CFTR, MUC5AC | |
| | TGFBR1, PDX1 | |
| | GNAS, TGFBR2 | |
| | (Ref. 29) | |
| 4 | Tp53 (Ref. 6) | miR-10b, miR-375, miR-101, miR-12, miR-130, miR- |
| EC | CDKN2A, | 143, miR-15a, miR-196b, miR-200a, miR-210, miR-223, |
| | CTNAP5, | miR27a, miR-28-3p, miR30b, miR-31, miR-454, miR-486, |
| | PIK3CA, SMAD4 | miR-574-3p, miR99b, miR-203, miR-146b, miR-21, miR- |
| | Amplified ERBB- | 197, miR-140, miR-96, miR-183, miR-205, miR-221, miR- |
| | 2, IHC3 | 503, miR-218, miR-124, miR-141, miR-220a, miR-214-3p, |
| | MET, EGFR, | miR-314a, miR-491, miR-203a, miR-506, miR-199a, miR- |
| | (Ref. 21) | 204, miR-328, miR-214, miR-126, miR-34a, miR-145 (Ref. |
| | TP53, EGFR | 7). |
| | ALDH2, PTGS2 | |
| | MET, CDKN1A | |
| | CD9, ADH1B | |
| | PIK3CA, PLCE1 | |
| | FGF4, CTTN | |
| | COL4A5, RB1 | |
| | CYP2A6, SOX2 | |
| | COL4A6, SPARC | |
| | CDC25B, FSCN1 | |
| | CRP, ADH1C | |
| | SPRR1A, NFE2L2 | |
| | (Ref. 30) | |
| 5 | EGFR, PIK3CA | Larynx: miR-21, miR-34a, miR-200c, miR-375, miR-23a, |
| HN | (Ref. 19) | miR-139-3p, mir885-5p, miR21-3p, miR-525-5p |
| | TP53 (Ref. 6) | Oral: let-7d, miR-218, miR-375, miR-10b, miR-21, miR- |
| | CDKN2A, GSTM1, | 196b, miR-494, miR34a, miR-340, mir-200a, miR-200b, |
| | GSTT1, PTGS2 | miR-200c, mir-429, mir-223 |
| | ALDH2, SDHD | Nasopharynx: miR-218, miR-17, miR-20A, miR-223, miR- |
| | SDHB, NOTCH1 | 29c |
| | TP63 , SDHC | Oropharynx: miR-21, miR-34a, miR-200c, miR275 (Ref. 7) |
| | ADH1B, MIR2 | |
| | CTTN, VHL | |
| | OGG1, ADH1C | |
| | NFIB, CKAP4 | |
| | CCNA1, PGLS | |
| | TNFRSF10B, | |
| | SDHAF2, S100A2 | |
| | ERCC4, XRCC3 | |
| | (Ref. 31) | |
| 6 | BRAF, EGFR, | let-7b, let-7c, let-7d, let-7e, let-7g, let-7i, miR-98, miR-17, |
| LC | KRAS, PIK3CA | miR-18a, miR-19a, miR-19b, miR-20a, miR-92a, miR-92a, |
| | (Ref. 19) | miR-205, miR-21, miR-1, mir-142-5p, miR-145, miR-34c, |
| | TP53 (Ref. 6) | miR-133b, miR-221, miR-222, miR-126, miR-212, miR- |
| | SCLC1 | 125a-3p, miR-125a-5p, miR-34a, miR-34b, miR-17-92, |
| | EGFR | miR-19a, miR-19b, miR-218, miR-7, miR-101, miR-451, |
| | TP53 | miR-192, miR-141, miR-200c, miR-21, miR-106a, miR- |
| | KRAS | 146b, miR-155, miR-17-5p, miR-221, miR-27a, miR-29c, |
| | ALK | miR-30a, miR-451, miR-449a, miR-449b, mIR-212, miR- |
| | MKI67 | 663, miR-29b, miR-22, miR-193b, miR-361-3p, miR-625, |
| | CDKN2A | miR-24, miR-30d, miR-196a, miR-99b, miR-203, miR- |
| | ERBB2 | 7515, miR-27a, miR-100, miR-186, miR-375, miR-138, |
| | PTEN | miR-31, miR-486-5p, miR-210, miR-365, miR-146a, miR- |
| | MET | 182, miR-197, miR-194, miR-148a, miR-497, miR-140, |
| | EML4 | miR-328, miR-339, miR-135b, miR-449a, miR335, miR- |
| | GSTM1 | 30d, miR-30e-5p, miR-24, miR-181b, miR-15, miR-205-3p, |
| | ERCC1 | miR-205-5p, miR-200a, miR-200a, miR-29a, miR-16, miR- |
| | CYP1A1 | 136, miR-9, miR-199a, miR-140miR 92b, miR-223, miR- |
| | AKT1 | 218, miR-198, miR-150, miR-1, miR-195, miR-503, miR- |
| | PROC | 545, miR-429, miR-93, miR-4782-3p, miR-142-3p, miR- |
| | BRAF | 9500, miR152, miR-183-3p, miR-95, miR-143miR-373, |
| | CTNNB1 | miR-153, mir-107, miR-203, miR-204, miR-217, mir-610, |
| | CCND1 | miR-96, miR-614, miR-338, miR-193a-3p, miR-124, miR- |
| | CD9 | 224, miR-204, miR-145, miR-25, miR-153, miR-137, miR- |
| | CASP3 | 148b, miR-152, miR-660, miR-181c, miR-181d, miR-331, |
| | KIT | miR-124, miR-526b, miR-342-3p, miR-99a, miR-512-3p, |
| | TNF | miR-1280, miR-129, miR-24, miR-5100, miR-146, miR-32, |
| | CDKN1A | miR-30d-5p, miR-154, miR-494, miR-15a, miR-675-5p, |
| | XRCC1 | miR-133a, miR-330-3p, miR-520a-3p, miR-370, miR-10b, |
| | (Ref. 32) | miR-494, mir-365, miR-570, miR-1469, miR-26b, miR-206, |
| | | miR-3662, miR-944, miR-106a, miR-203, miR- |
| | | 410miR1238, miR-503, miR-1246, miR-211, miR-224, |
| | | miR-497, miR-452, miR-498, miR-630, miR-365, miR-92a, |
| | | miR-564, miR-1290, miR-411, miR-93, miR-185, miR- |
| | | 1271, miR-16-5p, miR-15b-5p, miR-1246, miR-208a, miR- |
| | | 455, miR-377, miR-329, miR-652-3p, miR-509-5p, miR- |
| | | 1976, mir-1207, miR-1246, miR-1908, miR-320, miR-320a, |
| | | miR-424-3p, (Ref. 7) |
| 7 | PIK3CA (Ref. 19) | miR 155, miR 17-5p, miR-31, miR-125b, miR-183, miR- |
| BC | TP53 (Ref. 6) | 339-5p, miR-26a, miR-181a, miR-181b, mir-96, miR-126, |
| | BRCA2 | miR-495, miR-1258 (Ref. 7). |
| | BRCA1 | miRNA-7a, miRNA-7b, miRNA-7c, miRNA-7d, miRNA- |
| | MKI67 | 7e, miRNA-7g, miRNA-7i, miRNA-1, miRNA-100, |
| | TP53 | miRNA-107, miRNA-10a, miRNA- 10b, miRNA-122, |
| | ERBB2 | miRNA-1228, miRNA-124, miRNA-1258, miRNA-125a, |
| | CCND1 | miRNA-125b, miRNA-126, miRNA-127, miRNA-129, |
| | NODAL | miRNA-130a, miRNA-132, miRNA-132a, miRNA-133a, |
| | MUC1 | miRNA-135a, miRNA-139-5p, mir-140, mir-143, mir-145, |
| | CYP19A1 | mir-146a, mir-146b, mir-147, mir-148a, mir-148b, mir-149, |
| | PTEN | mir-150, mir-152, mir-153, mir-155, mir-15a, mir-16, mir- |
| | AKT1 | 16, mir-17, mir-17, mir-17-5p, mir-1 mir-181a, 81a, mir- |
| | PROC | 181b, mir-182, mir-1826, mir-183, mir-18a, mir-18b, mir- |
| | AR | 191, mir-193-a-3p, mir-193b, mir-195, mir-196a, mir-197, |
| | ABCB1 | mir-199b-5p, mir-19a, mir-19a-3p, mir-19b, mir-200a, mir- |
| | BIRC5 | 200b mir-200c, mir-203, mir-2004, mir-205, mir-206, mir- |
| | SRC | 20a, mir-20b, mir-21, mir-211, mir-216b, mir-217, mir-218, |
| | KITLG | mir-22, mir-221, mir-222, mir-224, mir-23a, mir-24, mir-24- |
| | CDKN1A | 2-5p, mir-24-3p, mir-26a, mir-26b, mir-27a, mir-29a, mir- |
| | CTNNB1 | 29c, mir-300, mir-30a, mir-31, mir-329, mir-335, mir-339- |
| | CHEK2 | 5p, mir-33b, mir-34a, mir-34b, mir-34c, mir-362-3p, mir- |
| | MYC | 373, mir-374a, mir-378, mir-379, mir-381, mir-383, mir- |
| | CDKN2A | 423, mir-425, mir-429, mir-450b-3p, mir-451a, mir-494, |
| | (Ref. 33) | mir-495, mir-497, mir-502-5p, mir-503, mir-510, mir-517a, |
| | | mir-519d, mir-520c, mir-526b, mir-574-3p, mir-638, mir- |
| | | 652-3p, mir-7, mir-720, mir-802, mir-873, mir-874, mir-92a, |
| | | mir-944, mir-96, mir-98, mir-99a, mir-290-3p, mir-290-5p, |
| | | mir-762, mir-27a, mir-671-5p, mir-340, mir-223, mir-506, |
| | | mir-33a, mir-129-5p, mir-381, mir-99a, mir-124, mir-204- |
| | | 5p, mir-675, mir-101, mir-2229, mir-520q, mir-449a, mir- |
| | | 498, mir-30a, mir-489, mir-34a, mir-21, mir-20a, mir-20b, |
| | | mir-489-5p, mir-625, mir-370, mir-892b, mir-145, mir-148a, |
| | | mir-142-3p, mir-195, mir-409-3p, mir-143, mir-let-7a, mir- |
| | | 5003-3p, mir-320a, mir-411-5p, mir-410-3p, mir-544, mir- |
| | | 3646, mir-146a, mir-485-5p, mir-485-3p, mir-30e, mir-421, |
| | | mir-490, mir-106b, mir-183-5p, mir-124-3p, mir-613, mir- |
| | | 214, mir-132, mir-212, mir-181-3b-3p, mir-544a, mir-601. |

1. SM: Sarcomatoid Mesothelioma;
2. GM: Glioblastoma- multiforme;
3. PC: Pancreatic Cancer;
4 EC: Esophageal Cancer;
5. HN: Head & Neck Cancer;
6. LC: Lung Cancer;
7. BC: Breast Cancer

DNA Damage Response (DDR)

Circulating cell free DNA from normal tissue (cDNA) and the cell free tumor DNA (ctDNA) increase several fold during radiation therapy (48). The ctDNA is mixed with cDNA but they can be differentiated. The circulating Epstein-Bar virus DNA (EBV DNA) in patients with nasopharyngeal cancer is mostly tumor derived. The surge of circulating EBV DNA in the first week of radiation therapy is more tumor specific (49). Circulating, mutated ctDNA is more tumors specific. Even more tumor specific is the circulating methylated DNA. Their kinetics is used for monitoring the tumor response to surgery and chemotherapy (50). In response to tumor DNA damaging cancer treatments, numerous DNA damage response (DDR) genes are activated. They include RADS, PARP1, BRCA1, ATM, TP53 (51) and others not yet defined. Mutated DDR genomes in tumor growth and metastasis are listed in Table 2.

TABLE 2
Potential Metastasis Causing Mutated
DNA Damage Response Gens

| No | Mutated Genome | Potential Metastasis |
|---|---|---|
| 1 | 14-3-3σ | Inactivated |
| 2 | ATM | Snail 1 phosphorylation andstabilization |
| 3 | BRCA1/BRCA2 | Down regulation |
| 4 | H2AX | Angiogenesis |
| 5 | MCPH1/BRIT1 | Expression |
| 6 | NBSI | Snail 1, MMP-2, EMT, migration, invasion, |
| | | metastasis |
| 7 | PARP-1 | Snail 1, transcription, EMT, invasion, metastasis |
| 8 | RAD 9 | Over expression |
| 9 | RAD 17 | Over expression |
| 10 | RAD 51 | May cause metastasis |
| 11 | TIP6 | Down regulation |
| 12 | TP53 | When mutated, promotes metastasis |
| 13 | XRCC3 | Invasion and metastasis |

In response to DNA damage, microRNA is modulated (52). DNA damage induced miRNA response can occur at both transcriptional and post-transcriptional levels. DNA damage response induced ATM also induce miRNAs possibly through p53 (53). Radiation induced DNA damage response is manifested by tumor suppressive and oncogenic mRNAs, microRNAs and proteins. They are carried to the receiving cells by exosomes (53). The molecular composition of such radiation enriched exosomes is altered. These exosomes are taken up by the receiving cells and they become migratory phenotypic cells that influence the tumor progression (53). Like the radiation enriched exosomes, hypoxia also enriches the exosomes in GBM cells. In vivo and in vitro, such hypoxia enriched exosome induce angiogenesis through phenotypic modulation of the tumor vascular endothelial cells. It activates the pericyte PI3K/AKT signaling and their migration. The hypoxic GBM exosomes accelerates the GBM tumor growth (54). Exosomes from patients with pancreatic cancer is loaded with mutated p53 and KRAS and double stranded DNAs (55). However, compared to RNA, the DNA contents in exosomes is very low (56).

The circulating DNA in plasma is protein-bound (nucleosomal) DNA. It has a short half-life (10 to 15 min). It is removed mainly by liver. Accumulation of DNA in circulation can result from an excessive release of DNA caused by massive cell death, inefficient removal of the dead cells, or a combination of both (54). Plasma levels of circulating cell free DNA (CCFDNA) does not change much in chronic kidney disease, peritoneal dialysis or hemodialysis (MB). However, it is removed by DCCFA, DPFA, DCFUC combined with SEC IEC and AC described in this invention.

Extracorporeal Chemotherapy Aided by Pulse-Flow-Continuous-Flow Ultracentrifugation and Chromatography

Surgery, radiosurgery, chemotherapy and chemo-radiosurgery causes surges of circulating DNA and DNA fragments (48. 49) and RNAs. It could lead to local tumor recurrence and abscopal metastasis (49B). It also induces abscopal tumor immunity (50, 57). DNA and DNA fragments repair after cancer treatments lead to increased tumor cell survival and diminished tumor control. The tumor growth enhancing repair of DNA/RNA, exosomes and microsomes after chemo-radiation could be controlled and inhibited by their extracorporeal treatments. The circulating tumor exosomes, microsomes and protein bound cell free DNA and DNA fragments and the RNAs are insufficiently inactivated by intracorporeal chemo-radiation therapy due to their toxicity and chemo-radioresistance. They could not be inactivated with in vivo maximum tolerable doses of radiation therapy and chemotherapy. It increases the recovery of the treated tumor cells. On the other hand if the intracorporeal radio-chemotherapy is supplemented with additional online extracorporeal chemotherapy EV labeled siRNA and chemotherapeutics that is sufficient to inactivate tumor specific exosomes, microsomes, micro RNAs and the protein bound DNA and DNA fragments, then the recovery of even the most chemo-radioresistant tumor is inhibited. It is accomplished in this invention by extracorporeal EV labeling with chemotherapeutics and returning such labeled EV to the patient instead of conventional direct administration of the chemotherapeutics to a patient. After extracorporeal EV labeling with siRNA and or chemotherapeutics by electroporation, by photochemical methods or using lipofectamine 2000, the labeled EV is returned to patient by EV infusion. Methods of extracorporeal EV labeling with siRNA is described in this invention. The EVs bind to their tissue specific targets through tissue specific receptors and adhesion molecules on their membrane. The EV-membrane-receptor-host cell-membrane bound EVs are taken up by the EV's cell of origin by phagocytosis or macropinocytosis and transfer their cargos into the host cells (4). It facilitates tumor specific chemotherapy with chemotherapeutics incorporated EVs prepared outside the patient without the need for direct administration of the chemotherapeutics to the patient. It mostly eliminates the systemic toxicity of the intracorporeal chemotherapy.

Metastasis Inhibiting Radiotherapy's EVs-AGO2-RISC-RAD51-diRNA-RNAi-miRNA, siRNA-Immunotherapy and Extracorporeal Chemotherapy Aided by Pulse Flow Apheresis and Continuous Flow Ultracentrifugation and Affinity Chromatography

Escape from innate immunity by circulating tumor cells and tumor cell derived extracellular vesicles (EVs) are among the primary causes of metastasis. Cancer treatments disseminate more cancer cells and EVs. The DNA damage repair response to radiation therapy releases extracellular vesicle-Agog-RISC-Rad51-diRNA-RNAi-siRNA-complexes. These complexes in turn induce systemic bystander effect innate immunity.

Radiation therapy changes the protein composition of the EVs. Genomic silencing with endogenous siRNA combined with radiation therapy's bystander effect does not affect innate immunity but it affects exogenous adaptive immunotherapy. Innate immunity is more effective than adaptive immunotherapy in protecting against hundreds of mutated proteins in a 100 to 1,000 nm sized EV.

Mutated genome silencing with endogenous RNAi-siRNA and miRNA with developing extracorporeal continuous flow, or pulse flow apheresis of cell bound proteomics and genomics, combined with molecular apheresis by sucrose density gradient (SDG) continuous flow ultracentrifugation (CFUC), and size exclusion-ion exchange chromatography improves innate immunity. Tumor-specific endogenous siRNA is generated from mutated RNA containing pre-miRNA hairpin through RNA induced silencing complex (RISC) composed of Dicer, dsRNA binding protein TRBP, and Argo2. Endogenous siRNA is generated by incubating purified RSIC with pre-let-7 hairpin which is then internalized into EVs and T-cells to silence its evasion from immunity by electroporation, by photochemical methods or using lipofectamine 2000. While undergoing radiation therapy, chemotherapy or surgery, a patient's blood is continuously drawn and processed through the above system. The entire plasma volume is treated several times until all or most of the mutated molecules are removed. After CFUC, sucrose is separated by cold sedimentation. The supernatant containing treated EVs with siRNA is returned to the patient. Phototherapy with internalized photosensitive complex-siRNA prevents development of chronic graft versus host disease of adaptive immunotherapy.

The clinical applications of radiation therapy's enhanced innate immunotherapy with purified EVs and T-lymphocytes incorporated with siRNA and virus apheresis are many They include virus removal and siRNA-silencing immunotherapy for lymphomas, leukemias, Merkel cell carcinoma, small cell lung cancer, aggressive triple negative breast cancer, pediatric diffuse infiltrating pontine glioma and glioblastoma multiforme, in addition to potential cure through siRNA induced neuron maturation. This potential, led by purified, mutated gene silencing with endogenous siRNA and miRNAs combined with radiation oncology's enhanced bystander effect innate immunity and extracorporeal chemotherapy cures more patients with cancer.

### BRIEF SUMMARY OF THE INVENTION

Radiation therapy changes the protein composition of EV. Genomic silencing with endogenous siRNA without adaptive response is an effective curative treatment for many cancers. It does not affect the innate immunity against changing protein composition in the EVs-exosomes as with adaptive immunotherapy. It adapts against hundreds of mutated proteins in a 100 to 1,000 nm-sized T-EVs-exosomes.

Mutated genome silencing with endogenous RNAi-siRNA and miRNA with near total cell and cell bound proteomics' apheresis by pulse flow apheresis and EV-exosome-RNA molecular apheresis by sucrose density gradient continuous flow ultracentrifugation combined with array centrifuge and continuous flow ultracentrifuge rotors and array centrifuge rotors molecular apheresis of both 50S and higher proteomics and genomics and 50S lower proteomics and genomics is disclosed. It purifies normal cell derived EVs-exosomes and T-EVs. They are fractionated into EVs and mutation specific T-EVs with immobilized Tim4-Fc protein Ca2+ magnetic beads affinity chromatography (ACG) and immobilized metal ACG. Tumor-specific endogenous siRNA is generated from mutated RNA containing pre-miRNA hairpin through RNA-induced silencing complex (RISC) composed of Dicer, dsRNA binding protein TRBP, and AGO2. Incubating purified RSIC with pre-let-7 hairpin generates siRNA. SiRNA is bonded with T-EVs and T-cells to silence its evasion from tumor immunity by electroporation, by photochemical methods, or with lipofectamine 2000. While on radiation therapy or surgery, a patient's blood is continuously processed through the above system as combined online radiotherapy, adaptive immunotherapy and tumor-seeking adaptive extracorporeal chemo-immunotherapy. The entire plasma volume in a patient is treated several times until all or most of the mutated molecules are removed. Plasma is cooled to 0° C. to prevent coagulation from heat generated by CFUC during centrifugation and to sediment sucrose after centrifugation. The supernatant containing treated T-EVs with siRNA and chemotherapeutics is returned to the patient. Near total treatment of immune cell and T-EVs by phototherapy with internalized photosensitive complex-siRNA cures immune-complex diseases and prevents chronic graft versus host disease.

This system's benefits include avoiding chemotherapy's bodily toxicity, virus removal, and tumor silencing siRNA-adaptive immuno-chemotherapy (siRAIC). It is used in treating lymphomas, leukemias, and Merkel cell carcinoma. SiRAIC is also used in treating lung cancer, breast cancer, pediatric diffuse intrinsic pontine glioma (DIP), in addition to potentially curing gliomas through siRNA induced neuron maturation. Mutated IDH1 and IDH2 molecular apheresis and silencing with siRNA prevent immune escape of gliomas, angioimmunoblastic T-cell lymphoma, acute myeloid leukemia, chondrosarcoma, and cholangiocarcinoma and other tumors.

### 1. BRIEF DESCRIPTION OF THE DRAWINGS

FIG. 24 shows removal of circulating tumor cells (CTC), RNA, DNA and DNA fragments, exosomes, microsomes and nanosomes from circulation.

FIG. 25A illustrates a continuous flow ultracentrifuge rotor adapted for plasmapheresis where filtered and cooled plasma from pulsed flow apheresis without macromolecules is made to flow through the bottom inlet of the rotor into a sucrose gradient solution for separation of normal cell derived nanomolecules, EVs-exosomes and tumor cell derived mutated nanomolecules and EVs-exosomes within the sucrose gradient by continuous flow ultracentrifugation.

FIG. 25B shows the same continuous flow ultracentrifuge rotor adapted for plasmapheresis of the pulsed flow apheresis plasma as illustrated in FIG. 25A but the supernatant exiting from the top hollow driveshaft 510 flows through two chromatography columns coated with patient specific tumor nanosomes antibody and connected with AFM, NTA, DCNA and a flow cytometer (FCM) for particle tracking and the effluent supernatant exiting from the chromatographic columns 534 flows back to the high speed rotating cylindrical rotor 508 through its bottom hollow driveshaft 502 and back to the patient or it re-circulates through a set of two affinity chromatography columns.

FIG. 25C illustrates the same continuous flow ultracentrifuge rotor adapted for plasmapheresis of the pulsed flow apheresis plasma as in FIG. 25A and FIG. 25B but the supernatant exiting from the top hollow driveshaft 510 flows through a series of chromatography columns and to nanosomes monitoring AFM, NTA, DCNA and FCM.

FIG. 25D illustrates a continuous flow ultracentrifuge rotor combined with a series of array rotors adapted for plasmapheresis of the pulsed flow apheresis plasma, its affinity chromatography and online monitoring of the subcellular EV-exosomes, DNA, and RNAs-proteomics during the treatment with biochemical testing devices and with AFM, NTA, DCNA and FCM.

FIG. 25E shows a continuous flow zonal rotor than those illustrated in FIG. 25A, FIG. 25B, FIG. 25C and FIG. 25D and it is combined with a series of array centrifuge and size exclusion chromatographic (SEC) and immuno-affinity chromatographic (IAC) and affinity chromatographic (AC) columns and AFM, NTA, DCNA and FCM for online monitoring of subcellular particles and EV-exosomes during radiation therapy and online extracorporeal chemotherapy.

### 2. REFERENCE NUMERALS

378. Corrugated pipe waveguide
380. Whole blood reservoir
382. Densitometer-1
384. Pulsed pump
386. CTC, plasma-platelet and exosomes, microsomes and nanosomes reservoir
388. Pulsed pump
390. Densitometer-2
392. DNA/RNA/Telomerase, exosomes, nanosomes affinity column-1 with EGCG
394. Densitometer-3
396. Pulsed pump
398. Purified plasma collecting bag
400. Densitometer-4
402. Reservoir with CTC, platelets, exosomes, microsomes and nanosomes/DNA-Telomerase
404. Pulsed pump
406. DNA/RNA/Telomerase, exosomes, nanosomes affinity column-2 with EGCG
408. Densitometer-5
410. Pulsed pump
412. Purified platelets collecting bag
414. Densitometer-6
416. Pulsed pump
418. Reservoir for RBC plus WBC and CTC, exosomes, microsomes and nanosomes
418B. Reservoir with WBC, CTC, exosomes, microsomes and nanosomes/DNA-Telomerase
420. Densitometer-7
422. Pulsed pump
424. DNA/RNA/Telomerase, exosomes, nanosomes affinity column-3 with EGCG
426. Densitometer-8
428. Pulsed pump
430. CTC, DNA/RNA/Telomerase, exosome, microsomes and nanosomes free WBC collecting bag
432. Densitometer-9
434. Pulsed pump
436. Reservoir with concentrated RBC, CTC, exosomes, microsomes and nanosomes/DNA-Telomerase
438. Densitometer-10
440. Pulsed pump
442. DNA/RNA/Telomerase, exosomes, nanosomes affinity column-4 with EGCG
444. Densitometer-11
446. Pulsed pump
448. Purified RBC collecting bag
450. Pulsed pump
452. Air bubble sensor
454. Densitometer-12
456. Treated return blood in blood flow tubing
458. Reservoir for DNA/RNA/Telomerase, tumor associated exosome, microsomes, nanosomes and CTC free blood after pulse flow purification
460. Blood flow inlet channel with clam and sensor
462. Blood flow return channel with clam and sensor
464. System clamp with sensors
466. Diluting normal saline
468. Anticoagulant reservoir
470. Blood flow tubing
472P. Microfilter for CTC removal from plasma
474P. Microfilter plasma CTC elution collection inlet and outlet
476P. Purified plasma collection inlet and outlet
476W Microfilter for removal of CTC bound to WBC
478R. microfilter for removal of CTC bound to RBC concentrate
478PL. Microfilter for removal of CTC bound to platelet
480PL. Microfilter platelet CTC elution collection inlet and outlet
482PL. Purified platelet collection inlet and outlet
484W. Microfilter WBC bound CTC elution collection inlet and outlet
486W. Purified WBC collection inlet and outlet
488R. Microfilter RBC bound CTC elution collection inlet and outlet
490R. Purified RBC collection inlet and outlet
492. Inlet and outlet tube connection
494. Ultracentrifuge continuous flow rotor
496. Bottom sample inlet
498. Mechanical seal
500. Damper
502. Bottom hollow driveshaft
504. Rotation Chamber
506. Core
508. High speed rotating cylindrical rotor
510. Top hollow driveshaft
512. High frequency motor
514. Top Mechanical seal
516. EV-exosome with siRNA effluent plasma flow towards chromatographic columns
517. EV-exosome with siRNA effluent plasma
518A. Flow control valve
518A2. Flow control valve
518B. SDG fraction collecting air injection
518C. SDG fraction collecting flow line
518C2. SDG fraction collecting flow line-2
518D. Affinity chromatographic column
518E. Purified SDG fraction flow to sucrose removing cold reservoir
518F. Purified SDG fraction collection line
518G. Purified SDG fraction collection line valve
518H. SDG fraction outlet and flow control valve
518-I. SDG fraction sample collection tubes
519. Processed plasma flow line
519B. Purifying effluent plasma
519C. Cooled sucrose free plasma
520. Sucrose removing cold reservoir
520B. Sedimented sucrose
521. Sucrose removed plasma with EV-exosome siRNA or chemotherapeutics
521B. cooling chamber
522. Processed plasma flow line
522A. Affinity chromatography column 1
522B. Affinity chromatography column-2
522C. Affinity chromatography column-3
522D. Affinity chromatography column-4
522E. Affinity chromatography column-5
522F. Affinity chromatography column-6
522G. Affinity chromatography column-7
522H. Affinity chromatography column-8
522I. Affinity chromatography column-9
522J. Affinity chromatography column-10
523. Purified, processed plasma with EV-exosome-siRNA-chemotherapeutics reservoir
524. Purified, processed plasma with EV-exosome-siRNA and or chemotherapeutics
525A. Purified, processed plasma with EV-exosome-siRNA-chemotherapeutics inlet to Patient
525B. Plasma inlet valve to patient
525C. Purified plasma with EV-exosome-proteomics-siRNA collecting bag
525D. Purified plasma with EV-exosome-proteomics-siRNA to collecting bag inlet valve
525E. Collecting bag's outflow
525F. Colleting bag's outflow controlling valve
526. Control system's LCD
522A. Affinity chromatography column-1
524B. Plasma injector to rotor
526B. Pulsed flow apheresis plasma into plasma injector
527A. Cooling chamber filter-1
527B. Cooling Chamber filter-2
528. Plasma injector
530A. Cooling Chamber-1
530B. Cooling Chamber-2
532. Warming coils
534. Effluent from the chromatographic columns
536. AFM
538. NTA
540. DCNA
542. FCM
544. Electronic flow direction control switch
546. Flow line for lab-testing
548. Array centrifuge
550. Titanium rotor for array centrifuge
552. Upper rotor half
554. Lower rotor half
556. Array centrifuge rotor shaft
558. Electronic inlet and outlet flow directing switch
560. O-ring
562. Rotor spin rate controlling computer
564. Disposable polypropylene rotor inserts
565 Polyethylene rotor wall
568. Directional arrows
570. SDG fraction collecting flow line
572. Electronic flow control valve
574. Portable trailer
576. Continuous flow rotor
578. Plasma flow control valve
580. Rotating seal assembly
582. Central inlet
584. Upper radial channel
585. Air block
586. Inner surface of the bowl wall
588. Core tapper volume
590. Core
592. Connecting channel to central flow
594. Edgeline outlet
596. SDG fraction flow to photometer/flow cell
598. Photometer/Flow cell
600. SDG Fraction collector
602. Lid
604. Bowl
606. Buffer reservoir
608. SDG solution reservoir
610. Buffer line
612. SDG line
614. Buffer and SDG flow line valve
616. Displacement dense fluid reservoir
618. Displacement dense fluid flow line

### 3. DETAILED DESCRIPTION OF THE PREFERRED EMBODIMENTS

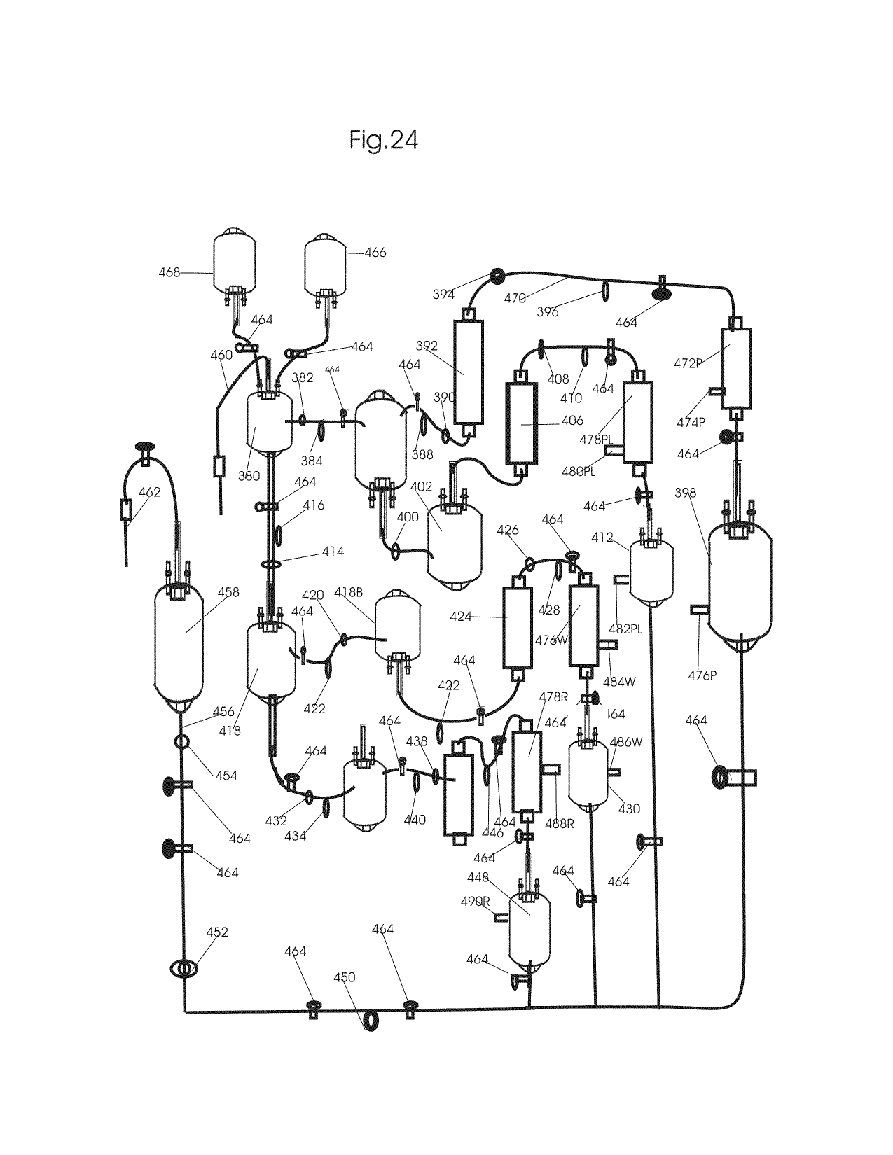

FIG. 24 shows removal of circulating tumor cells (CTC), RNA, DNA and DNA fragments, EVs-exosomes, microsomes and nanosomes from circulation after cancer treatments by pulsed flow apheresis to minimize mutated gene induced bystander and abscopal effects associated tumor recurrence and metastasis. In this invention, tumor cell derived mutated subcellular components is removed by a pulse flow system combined with DNA-siRNA-affinity chromatography. Two intermittent pulse flow apheresis systems are run simultaneously to have a continuous flow apheresis of the EVs-exosomes, microsomes nanosomes. One of such intermittent pulse flow system is shown in FIG. 24. It consists of the whole blood reservoir 380 to which the whole blood drawn from the patient at a rate of 15 to 150 ml/min through the blood flow inlet channel with clam and sensor 460 is collected. After drawing about 300 ml blood, the blood flow to the whole blood reservoir 380 is stopped by clamping the clamp with air and pressure sensors 464A and 464B. The whole blood drawn is then mixed with anticoagulant to keep the blood from clotting and to keep the blood at its normal viscosity from the anticoagulant reservoir 468 and normal saline from the normal saline reservoir 466 if needed to adjust the hematocrit reading. By 15 min gravity sedimentation the plasma layer with platelets and at its bottom the heavier white blood cells, the red cells and the very bottom circulating tumor cells (CTCs) if any are separated. A series of system clamps with air and pressure sensors, 464, a series of densitometers, the densitometer 382, 390, 394, 400, 408, 414, 418, 420, 426, 432, 438, 444 and 454, a series of pulsed pumps, pulsed pump 384, 388, 396, 404, 410, 416, 422, 428, 434, 440, 446 and 450, whole blood and EVs-exosomes reservoir 380 the plasma-platelet and EVs-exosomes, microsomes and nanosomes reservoir 386, reservoir for RBC plus WBC, CTC, EVs-exosomes, microsomes and nanosomes 418, to the reservoir with WBC, CTC, EVs-exosomes, microsomes and nanosomes/DNA-DNA-fragments and RNAs 418B, and to the reservoir for concentrated RBC and CTC, EVs-exosomes, microsomes, nanosomes and CTC, DNA-DNA fragments 436, separate reservoir with platelets, exosomes, microsomes and nanosomes/DNA-DNA fragments RNAs 402, reservoir with WBC, EVs-exosomes, microsomes and nanosomes/DNA-DNA fragments, RNAs 418B, reservoir for concentrated RBC and extracellular vesicles-exosomes, microsomes, nanosomes CTC, DNA, DNA-fragments and RNAs 436, a series of DNA/RNAs siRNA-affinity columns, DNA/RNA/RNAs, exosomes, nanosomes siRNA-affinity column-1 392, DNA/RNA/extracellular vesicles-exosomes, nanosomes siRNA-affinity column-2, 406, DNA/RNA/RNAs, extracellular vesicles-exosomes, nanosomes siRNA-affinity column-3, 424 and DNA/RNA/RNAs, extracellular vesicles-exosomes, nanosomes siRNA-affinity column-4, 442, a series of microfilters for CTC removal from plasma 472P with microfilter plasma CTC elution collection inlet and outlet 474P, microfilter for removal of CTC bound to platelet 478PL, with microfilter platelet CTC elution collection inlet and outlet 480PL, microfilter for removal of CTC bound to WBC 476W with microfilter WBC bound CTC elution collection inlet and outlet 484W, microfilter for removal of CTC bound to RBC concentrate 478R with microfilter RBC bound CTC elution collection inlet and outlet 488R, a series of processed blood components collecting bags with siRNAs, semi-purified plasma collecting bag with siRNAs 398 with semi-purified plasma collection inlet and outlet 476P to remove samples of treated plasma for testing and preservation before its transfusion back to the patient or continuous flow ultracentrifugation plasmapheresis, purified platelets collecting bag with siRNAs 412 with purified platelet collection inlet and outlet 482PL to remove samples of treated platelets for testing before its transfusion back to the patient or preservation, CTC, DNA/RNA-EVs-exosome, microsomes and nanosomes WBC treated with siRNA 430 with purified WBC collection inlet and outlet 486W to remove samples of purified WBC for testing before its transfusion back to the patient or preservation, purified RBC collecting bag with siRNA 448 with purified RBC collection inlet and outlet 490R to remove samples of treated RBC for testing and preservation before its transfusion back to the patient or preservation, blood flow tubing 470 which interconnects blood and blood component reservoirs and with the reservoir for DNA/RNA/EVs-exosome, microsomes, nanosomes and CTC free blood after pulse flow purification 458.

CTC separation by microfiltration is fast and simple. After chemotherapy/radiosurgery large volumes of blood apheresis is processed rapidly to remove CTC, CTC-bound to platelets, exosomes, microsomes and nanosomes and to remove the DNA-DNA fragments, and RNAs. Over 90 percent of CTC can be removed by rapid CTC microfiltration (63). Selected in-vitro and in-vivo methods of EVs-exosome, RNA, DNA cellular interaction described in the literature is used to test patient specific EVs-exosome, RNA and DNA interactions. They are listed below. The blood components are passed thorough siRNA affinity chromatograms Heparin mimics as a DNA binding polyanionic structure nucleic acid (65) Partial purification of DNA binding proteins with HiTrap heparin column is commercially available (66)Cellulose activated charcoal coated with heparin is safely used in hemoperfusion for drug overdose treatment (67). Disposable DNA/RNA/EVs-exosomes, microsomes and nanosomes binding heparin coated cellulose activated charcoal is used to remove the DNA/RNA/EVs-exosomes, microsomes and nanosomes surge caused by chemotherapy-radiosurgery and surgery and or siRNA incorporation into EVs-exosomes. It eliminates and or minimizes the bystander and abscopal effects associated metastasis from mutated genomes.

Air bubble sensor 452 monitors any air bubbles in the final stretch of the blood flow tubing 470 that connects with the reservoir for DNA/RNA/, tumor associated EVs-exosomes, microsomes, nanosomes and CTC free blood after pulse flow purification 458. If there are air bubbles, they are purged out of the blood flow tubing 470 by opening and closing the system clamps with sensors 464 adjacent to the reservoir for DNA/RNA/, tumor associated EVs-exosomes, microsomes, nanosomes silenced with siRNA 458 The CTCs are filtered out. The densitometer-12 454 monitors the siRNA treated return blood cellular elements diluted with saline in blood flow tubing 456. The purified blood cellular element treated with siRNA and diluted with saline is transfused back to the patients through blood flow return channel with clam and sensor 462. The separated plasma containing DNA/RNAs and exosomes is further treated in DCFUC and array centrifuges combined with size exclusion chromatographic columns (SEC) and immuno-affinity chromatographic columns, (IAC) and affinity chromatography (AC). After apheresis of about 300 ml with the first apheresis system is completed, apheresis with the second set of the pulse flow apheresis system is started by connecting it to the patient at another blood drawing site; say to the left arm if the first pulse flow apheresis system was connected to the right arm. Intermittent apheresis with two such systems facilitates a continuous pulse flow aphaeresis.

The pulse flow apheresis of cell bound proteomics and genomics combined with siRNA affinity chromatography and molecular apheresis by SDG continuous flow ultracentrifugation improves innate immunity. Tumor-specific endogenous siRNA is generated by incubating purified RSIC with pre-let-7 hairpin. It is internalized into EVs and T-cells by electroporation, by photochemical methods or using lipofectamine 2000. They silence the evasion of immune cells from immunity and inhibit tumor recurrence and metastasis.

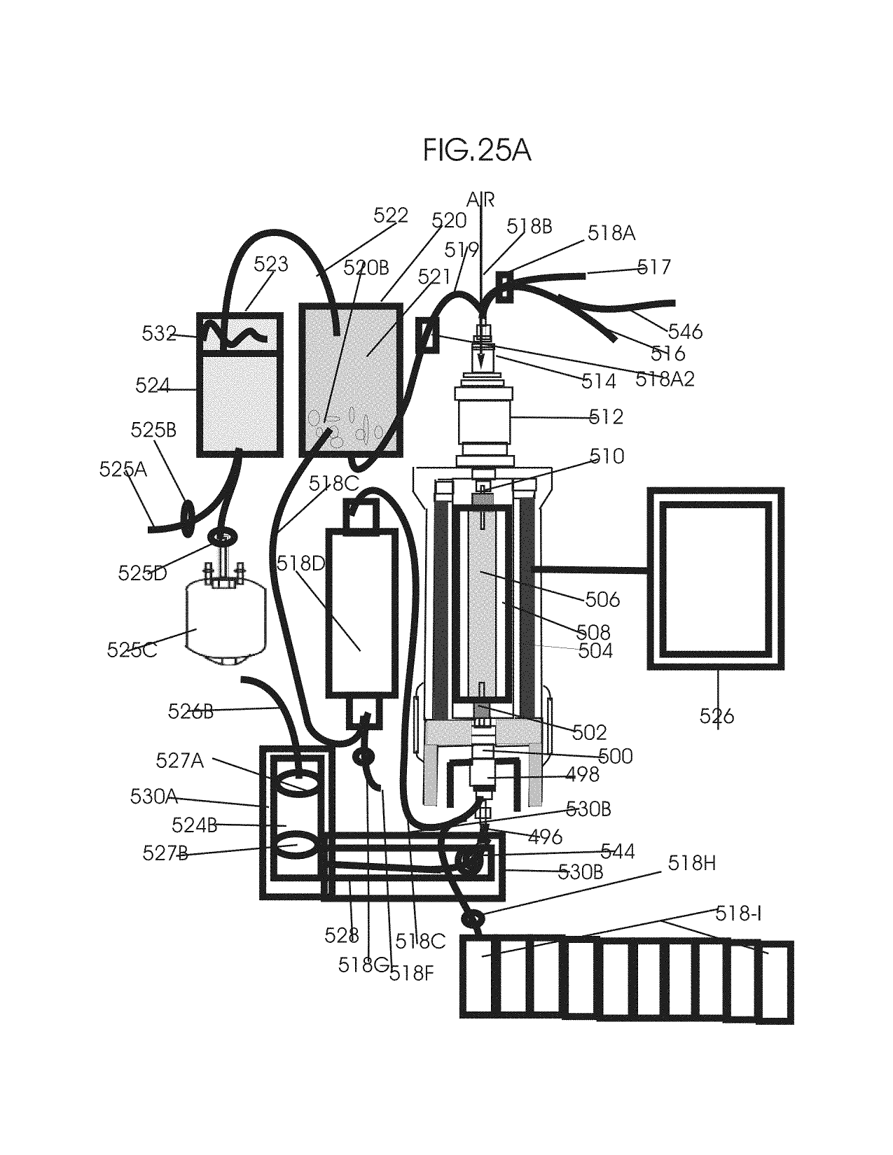

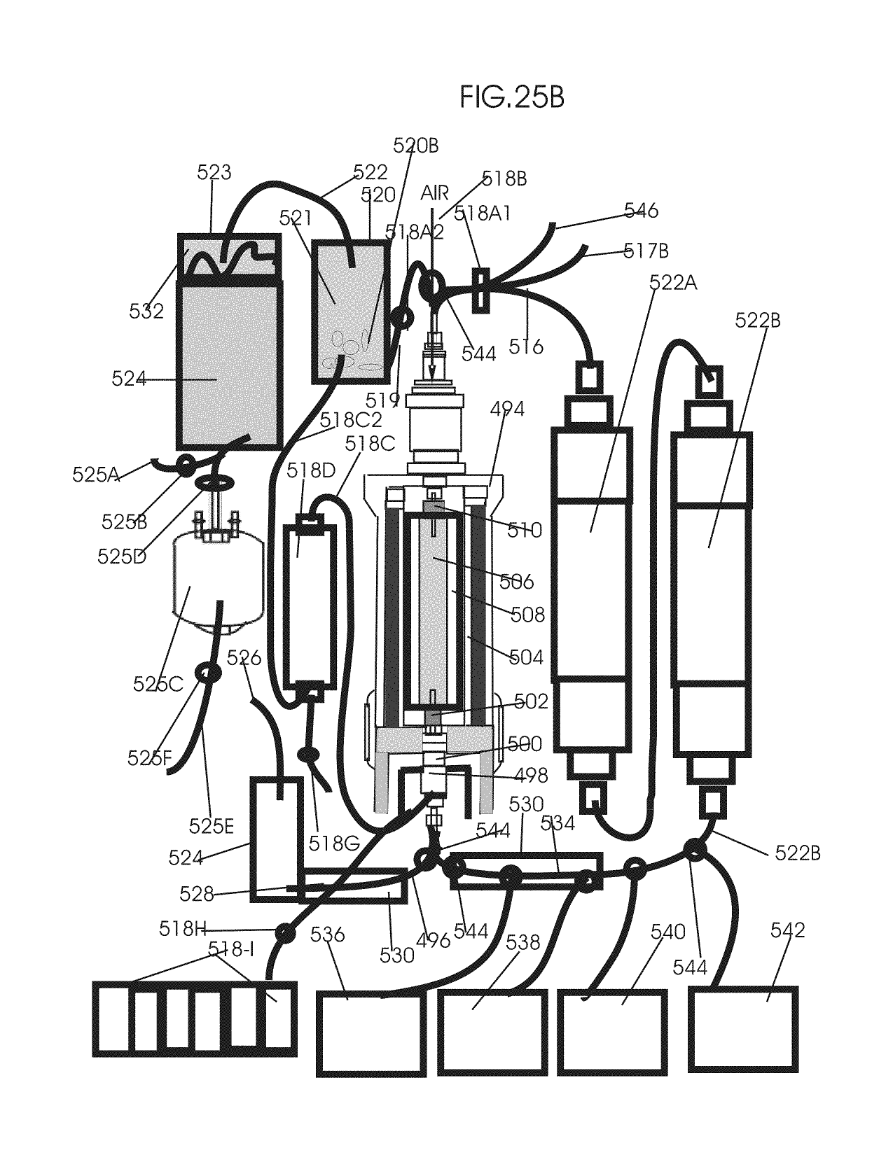

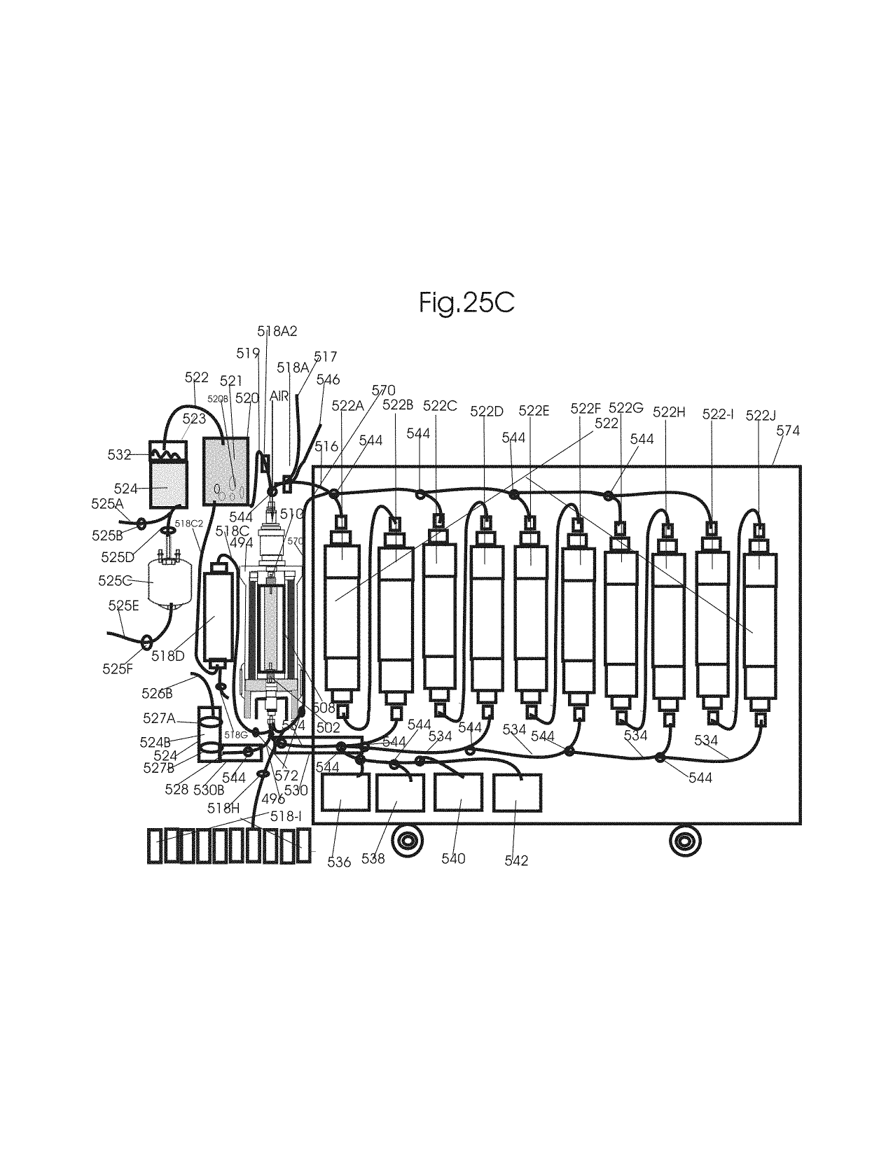

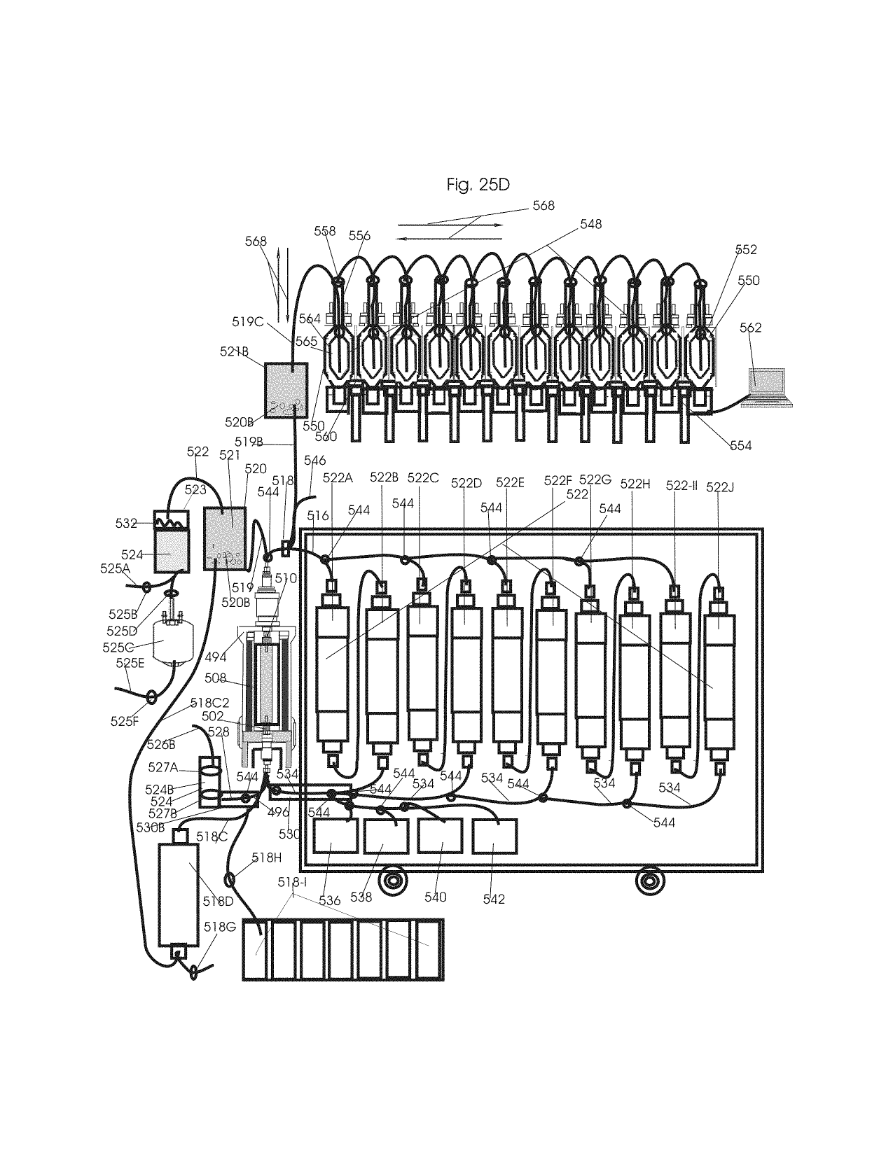

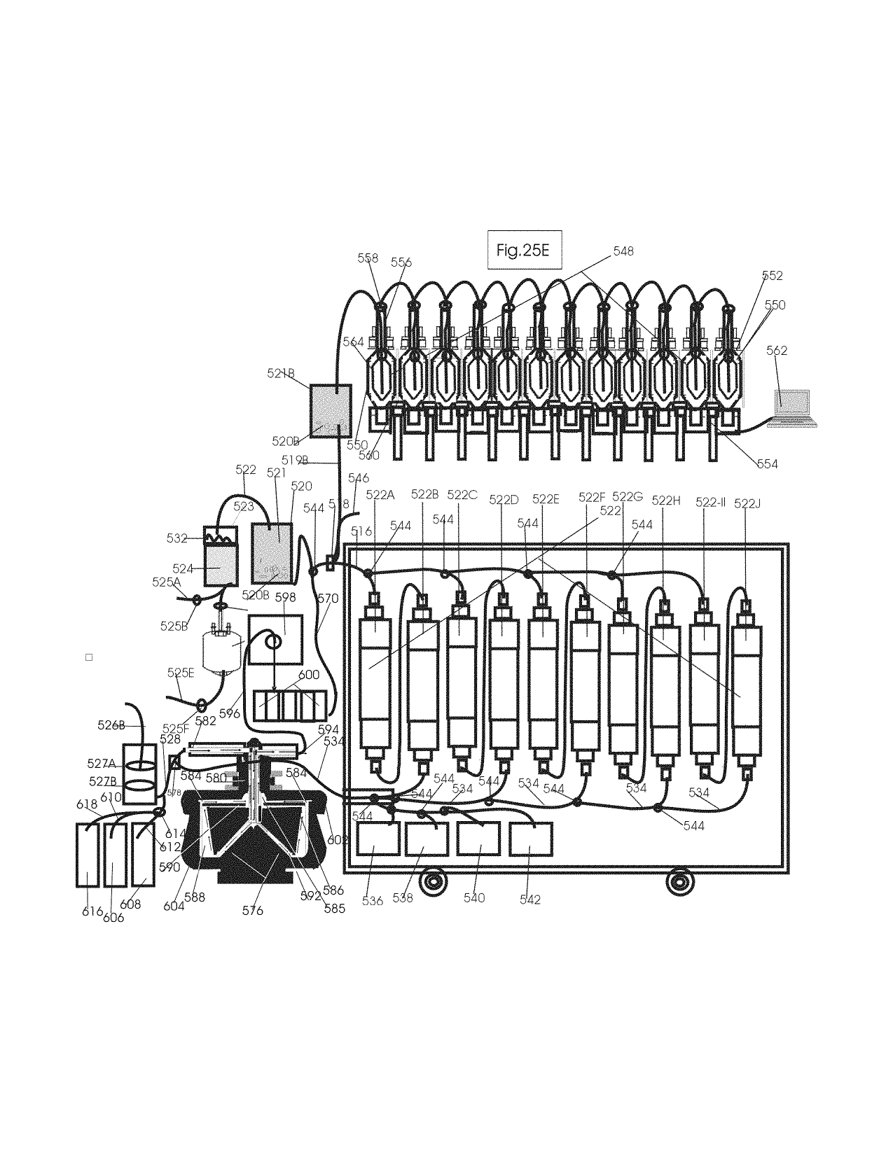

FIG. 25A illustrates a continuous flow ultracentrifuge rotor adapted for plasmapheresis where filtered and cooled plasma from pulsed flow apheresis without macromolecules is made to flow through the bottom inlet of the rotor into a sucrose gradient solution for separation of normal cell derived nanomolecules, EVs-exosomes and tumor cell derived mutated nanomolecules and EVs-exosomes. They separate within the sucrose gradient by continuous flow ultracentrifugation. Plasma nanomolecules reaming in the rotor and flowing with effluent plasma through the top rotor outlet is controlled by controlled rotor speed and plasma flow rate. The rotor outlet effluent plasma with EVs-exosomes flows towards Tim4-Fc magnetic beads columns for additional purification. The immobilized Tim4-Fc protein Ca2+ magnetic beads affinity column (ITMAC) captures the EV-exosomes. The purified EVs-exosomes is eluted from the ITMAC with EDTA (71, 72). It is followed by additional purification and separation with a series of immobilized metal affinity chromatographic columns (IMAC) or other suitable chromatographic columns selected from the group consisting of size exclusion chromatographic columns (SEC), immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. Based on patient specific micro and nano-molecule's separation, specific chromatographic columns are selected. Such chromatographic columns are routinely used in virus purifications (73). By adjusting the rotor speed and flow rate and depending on the size, shape and densities, isopycnic banding, the differential fractionation of the plasma soluble and plasma suspended subcellular molecules are collected by SDG ultracentrifugation. The higher molecular weight and size subcellular fractions are sedimented within the sucrose density gradient in the rotor. The lower and lowest molecular weight subcellular molecules flow through the top rotor outlet with the plasma effluent. At the end of the ultracentrifugation, the speed of the rotor is slowly reduced to 4,000 rpm/min and slowly brought to stop. Fractions of the SDG are collected by air injection through the top hollow driveshaft 510.

The continuous flow ultracentrifuge with continuous flow rotors are generally used to separate micro and nano particles in nanoparticle research and industry. In pharmaceutical industry, they are used to produce vaccines. For the purpose of illustration, such a continuous flow ultracentrifuge rotor made by Hitachi Koki Co. Ltd is described herein in its entirety (26) but with adaptive modifications to suite the removal of remaining nanoparticles after pulse flow apheresis of plasma and its filtration. Any other continuous flow ultracentrifuge and continuous flow rotors could be modified and adapted for molecular apheresis and purification and separation of normal cell derived and tumor cell derived DNA, RNAs, EVs-exosomes nanosomes, and ribosomes in the plasma after pulse flow apheresis. The continuous flow ultracentrifuges and rotors that are suitable for such adapted use include Alpha Wassermann continuous flow ultracentrifuge and rotors, Beckman continuous flow ultracentrifuge and rotors, Sorvall-Thermo Fisher continuous flow ultracentrifuge and rotors or any other similar ones from any other manufacturers. They are also adapted to use with array centrifuge taught in U.S. Pat. No. 6,387,031 which is described herein in its entirety (74) but with adaptive modifications to suite the biochemical testing and removal of still remaining EV-exosome-proteomic nanoparticles after pulse flow apheresis of plasma and its filtration and by continuous flow SDG ultracentrifugation that also separates and removes the subcellular molecules.

As part of the EV-exosome-proteomic nanoparticle separation and apheresis combined mutated gene silencing with siRNA and extracorporeal chemotherapy, the pulsed flow apheresis plasma is continuously introduced into the high speed rotating cylindrical rotor 508 through its bottom sample inlet 496. High speed rotating cylindrical rotor 508 is connected to the hollow top driveshaft 510 and to the bottom hollow drive shaft 502 for the sample to pass through and are supported by bearings. The driveshaft at the top is connected to a high frequency motor 512. The mechanical seal at the end of the upper driveshaft 502 and the bottom driveshaft 510 seals the sample from any leaks. The rotating cylindrical rotor 508 rotates at any adjustable speeds as desired up to 40,000 rpm/min, 100,000 G and volume. By adjusting the rotor speed and flow rate, plasma subcellular molecules are removed either with the sucrose density gradient in the rotor as SDG fractions or as EV-exosome with siRNA effluent plasma flow towards chromatographic columns 516 (not shown) or as EV-exosome with siRNA and chemotherapeutics containing effluent plasma 517 that flows towards array centrifuges (not shown) that is controlled by the flow control valve 518. After plasma is processed, the purified processed plasma with EV-exosome—siRNA and sucrose flows to sucrose removing cold reservoir 520 through processed plasma flow line 519. Sucrose removed plasma with EV-exosome siRNA or chemotherapeutics 521 flows through processed plasma flow line 522 to purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A or it is collected into the collecting bag 525C by closing the plasma inlet valve to patient 525B and letting the flow of the purified plasma with EV-exosome and proteomics by opening the purified plasma with EV-exosome-proteomics-siRNA to collecting bag inlet valve 525D. The purified EV-exosome-proteomics-siRNA is collected into purified plasma with EV-exosome-proteomics-siRNA collecting bag 525C. It is transfused back to the patent using the collecting bag's outflow 525E with colleting bag's outflow controlling valve 525F. The operation parameters of the ultracentrifuge with the rotor including electrical, cooling, vacuum and the mechanical seal and status of the motor, are displayed on the control system LCD 526.

The sucrose gradient solution consisting of 130 ml phosphate buffered saline, 200 ml 17% (W/W) sucrose (density 1.0675 g/cm²), 130 ml 30% (W/W) sucrose (density 1.1.1268 g/cm²) and 30 ml 45% (W/W) sucrose (density 1.2028 g/cm²) (85) is filled into the rotor that can hold about 3 L fluid. Any other small volume rotors and SDG concentration suitable for medical application could also be used. The sucrose gradient solution is filled into the rotor through the bottom hollow driveshaft 502 and the centrifuge is run at 4,000 rpm/min for a few minutes to layer the sucrose gradient solution vertically. It causes the higher concentration sucrose solution to migrate towards the center of the rotor and the lower concentration to move towards the periphery of the rotor forming a density gradient between these layers. After the density gradient is formed, the filtered and cooled pulse flow apheresis plasma without macromolecules from pulse flow plasma cooling chamber 530A and pulse flow plasma cooling chamber 530B is injected into the rotor through the bottom hollow driveshaft 502 at an injection rate of about 5-20 ml/min while the rotor is slowly accelerated to desired speed to separate intended plasma suspended micro or nanomolecules. Before injecting the pulse flow apheresis plasma into the rotor, it is chilled to about 0° C. in the cooling chambers. Cooled-filtered pulse flow apheresis plasma is injected into the rotor through plasma injector 528. The additional cooling is an added precaution to prevent plasma coagulation from the heat generated by the rotation of the rotor. The slow flow rate and high speed rotation of the rotor maintains the sucrose gradient undisturbed (68). Plasma volume for an adult is about 3 L. (69) which is constantly monitored by bioelectrical impedance analysis (BAL) (91X) (70) and maintained at this total body plasma volume with 5% D/0.45 N saline supplemented with electrolytes like potassium, calcium, magnesium if needed to maintain patient's electrolytes and fluid balance. The electrolyte levels are constantly monitored. Continuous plasmapheresis at a rate of about 5-20 ml/min will complete one course of 3 L plasmapheresis within about 10 or 2 hours. In general when continuous flow centrifuges (not the ultracentrifuge) are used for blood component exchanges, the usual flow rate is 40 ml/min (71). Since the pulse flow apheresis system is not based on centrifugation, its flow rate is slower. Safe centrifugal apheresis at rate of 50-150 ml/min is in common practice (72). Because of the intermingling of the plasma with other body fluid compartments, a one or two times whole body plasma aphaeresis is not sufficient to remove and process all the circulating mutated cancer molecules suspended in the whole body plasma. The rate of clearance of the tumor associated mutated nanoparticle in the plasma is monitored with AFM, NTA and DCNA, flow cytometry and other testing listed According to the size and weight of the nanoparticles in the pulsed flow apheresis plasma, they separate towards the inside of the sucrose gradient solution. At the end of the ultracentrifugation, the speed of the rotor is slowly reduced to 4,000 rpm/min and slowly brought to stop. Fractions of the SDG are collected by air injection through the top hollow driveshaft 510.

The continues-flow ultracentrifuge rotor is run at elective speed and g-force from 50,000 to 100,000 g for about 12 hrs at 4° C. for elective fractionation of plasma soluble and suspended EV-exosomes, exRNAs, tRNAs, DNA fragments, and tumor associated proteins like IDH1 and IDH2 and virus and virus fragments. The rate of clearance of these particles is monitored with AFM, NTA and DCNA and flow cytometry (not shown here). At the end of this ultracentrifugation, the particle that layers in sucrose density gradient contains most of the larger plasma soluble and suspended particles.

The effluent through the top of the rotor outlet contains plasma soluble and suspended nanoparticles which exit from the top hollow driveshaft 510 of the rotor is directed towards a series of chromatographic columns through EV-exosome with siRNA effluent plasma flow towards chromatographic columns 516 or to EV-exosome with siRNA effluent plasma 517 that flows towards array centrifuges (not shown here) through flow control valve 518A. The flow-line 546 is connected with biochemistry testing devices. The immobilized Tim4-Fc protein Ca2+ magnetic beads affinity column and the series of immobilized metal affinity chromatographic columns and other suitable chromatographic columns selected from the group described above purifies and separates the normal cell derived EVs and the tumor cell derived mutated EVs. The purified EVs-Exosomes are labeled with siRNA and or with chemotherapeutics by electroporation, or by photochemical methods or with lipofectamine 2000. Such labeled EVs containing siRNA and or chemotherapeutics and sucrose gradient plasma is cooled to Oo C in sucrose removing cold reservoir 520 where sedimented sucrose 520B collects at the bottom. The supernatant plasma with EV-exosome-siRNA is warmed to 370 C with a warming coil 532 in purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A. Alternatively, the purified plasma with EV-exosome-proteomics-siRNA flow valve 525D is opened and the processed plasma with EV-exosome-siRNA and or chemotherapeutics 524 is collected into purified plasma with EV-exosome-proteomics-siRNA collecting bag 525C and preserved for its biochemical analysis and or later administration to the patent.

The subcellular fractions and the EV-exosome-separated into sucrose gradient cushion are collected from the bottom of the high speed rotating cylindrical rotor 508. The rotational rpm is reduced to 4,000 and the rotor is brought to a halt without disturbing the sucrose density gradient. After rotor comes to a standstill, flow control valves 518A and 518A2 are closed and air is injected into the rotor through the top 0f the rotor to push the SDG layers containing subcellular DNA, RNA, extracellular vesicles-exosomes and nanosomes. Based on molecular weight configuration of the particles and sedimentation coefficients higher than 50S, they sediment into varying layers of the sucrose gradient. Particles with less than 50 S sedimentation coefficients are not suitable for separation by continuous flow ultracentrifugation (75). Subcellular DNA, RNA, extracellular vesicles-exosomes and nanosomes with higher than 50 S sedimentation coefficient separated into glucose density gradient by continuous flow ultracentrifuge with high speed rotating cylindrical rotor 508 flows through SDG fraction collecting flow line 518C to affinity chromatographic column 518D. SDG fractions are also collected per SDG fraction sample outlet and flow control valve 518H into SDG fraction collection tubes 518-I for biochemical testing and to check integrity of EV-exosomes including testing for CD9, CD63, CD73 and CD90, and their size and morphology. SDG fraction collecting flow line 518C leads SDG fraction per fraction to affinity chromatographic column 518D. Only one such affinity chromatographic column 518D is illustrated here but it can be multiple as shown in FIG. 25B, FIG. 25C, FIG. 25D and in FIG. 25E. As described before, the affinity chromatographic columns selected include immobilized Tim4-Fc protein Ca2+ magnetic beads affinity column (ITMAC), iZON science's size exclusion chromatographic columns (SEC) qEV, qViro-X or from a series of immobilized metal affinity chromatographic columns (IMAC) or other suitable chromatographic columns selected from the group consisting of other types of SEC, immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. Based on patient specific micro and nano-molecule's separation need, specific chromatographic columns are selected. Such chromatographic columns are routinely used in virus purifications (73). Tumor-specific endogenous siRNA is generated from mutated RNA containing pre-miRNA hairpin through RNA induced silencing complex (RISC) composed of Dicer, dsRNA binding protein TRBP, and Argo2 (76). It is generated by incubating purified RSIC with pre-let-7 hairpin (76). This siRNA is then internalized into EVs and into T-cells by electroporation (77) or by photochemical methods or with lipofectamine 2000. Such T-cell treatments silence its evasion from immunity. Chemotherapeutics are also internalized into EVs by electroporation, photochemical methods or with lipofectamine 2000. Labeled EVs containing siRNA and or chemotherapeutics and sucrose gradient plasma flows to sucrose removing cold reservoir 520 where it mixes with effluent plasma existing from the top of the rotor 514. It is cooled to 0° C. to sediment sucrose and the sedimented sucrose 520B collects at the bottom. As with the effluent plasma existing from the top of the rotor 514 the SDG fraction's purified plasma with EV-exosome-siRNA with or without chemotherapeutics is warmed to 37° C. with a warming coil 532 in purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A. Alternatively, the purified plasma with EV-exosome-proteomics-siRNA flow valve 525D is opened and the processed plasma with EV-exosome-siRNA and or chemotherapeutics 524 is collected into purified plasma with EV-exosome-proteomics-siRNA collecting bag 525C and preserved for its biochemical analysis and or later administration to the patent. The continuous flow ultracentrifuge and all its accessories are kept in sterile conditions in a sterile environment. The rotor is sterilized online as per manufacturer's instructions. It is also kept sterile and operated in sterile conditions.

FIG. 25B shows the same continuous flow ultracentrifuge rotor adapted for plasmapheresis of the pulsed flow apheresis plasma as illustrated in FIG. 25A but the supernatant exiting from the top hollow driveshaft 510 flows through two affinity chromatography columns and connected with AFM, NTA, DCNA and a flow cytometer (FCM) for particle tracking and the effluent supernatant exiting from the chromatographic columns 534 flows back to the high speed rotating cylindrical rotor 508 through its bottom hollow driveshaft 502 and back to the patient or it re-circulates through a set of two affinity chromatography columns.

For separation of patient specific EV-exosomes and plasma soluble and plasma suspended subcellular micro and nano particle, and proteomics the EV-exosome with siRNA effluent plasma flow towards chromatographic columns 516 exiting from the top hollow driveshaft 510 is directed towards chromatography column 1, 522A and to chromatography column 2, 522B. Alternatively, this EV-exosome with siRNA effluent plasma with EV-exosomes, siRNA and or chemotherapeutics and sucrose gradient is directed towards array centrifuges 517B that is controlled by the flow control valve 518 (array centrifuge is not shown). The flow-line 546 is connected with biochemistry testing devices. The affinity chromatographic columns selected include Tim4-Fc magnetic beads columns, immobilized Tim4-Fc protein Ca2+ magnetic beads affinity column (ITMAC), iZON science's size exclusion chromatographic columns (SEC) qEV, qViro-X, immobilized metal affinity chromatographic columns (IMAC), size exclusion chromatographic columns (SEC), immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. The chromatographic columns are selected on the basis of patient specific subcellular fractions present in the plasma effluents.

As described under FIG. 25A, after plasma is processed, the purified processed plasma with EV-exosome-siRNA and sucrose flows to sucrose removing cold reservoir 520 through processed plasma flow line 519. Sucrose removed plasma with EV-exosome siRNA or chemotherapeutics 521 flows through processed plasma flow line 522 to purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A or it is collected into the collecting bag 525C by closing the plasma inlet valve to patient 525B and letting the flow of the purified plasma with EV-exosome and proteomics by opening the purified plasma with EV-exosome-proteomics-siRNA to collecting bag inlet valve 525D. The purified EV-exosome-proteomics-siRNA is collected into purified plasma with EV-exosome-proteomics-siRNA collecting bag 525C. It is transfused back to the patent using the collecting bag's outflow 525E with colleting bag's outflow controlling valve 525F.

The subcellular EVs-exosomes, genomics and proteomics are monitored with AFM 536, NTA 538, and DCNA 540 and to a flow cytometer (FCM) 542 for particle tracking. The effluent supernatant exiting from the chromatographic columns 534 flows back to the high speed rotating cylindrical rotor 508 through its bottom hollow driveshaft 502 and back to the sucrose removing cold reservoir 520 through processed plasma flow line 519 or it re-circulates through the chromatography column1 522A and chromatography column2, 522B through the supernatant outlet 516. Before the effluent supernatant exiting from the chromatographic columns 534 is injected back into the high speed rotating cylindrical rotor 508, it is cooled to 0° C. with the cooling coil 530. The supernatant flow into the rotor, out of the rotor, into the chromatography columns, into AFM, NTA, DCNA and FCM and back to the reservoirs or to the chromatographic columns is controlled by the electronic flow direction control switch 544. Before the effluent supernatant exiting from the high speed rotating cylindrical rotor 508 is returned back to patient, it is warmed to w 37° C. with the warming coil 532. Before the pulsed flow apheresis plasma is injected into the high speed rotating cylindrical rotor 508 through its bottom hollow driveshaft 502 for nanoparticles separation, it is cooled to 0° C. with the cooling coil 530. The chromatography columns are sterilized and kept in a sterile environment. The continuous flow ultracentrifuge is also kept in sterile condition and the rotor is sterilized online as per manufacturer's instructions. It is kept sterile and operated in sterile conditions.

The extracorporeal continuous flow, or pulse flow apheresis of cell bound proteomics and genomics, combined with molecular apheresis by sucrose density gradient continuous flow ultracentrifugation, and chromatography with siRNA affinity columns, Tim4-Fc magnetic beads columns, immobilized Tim4-Fc protein Ca²⁺ magnetic beads affinity column, immobilized metal affinity chromatographic columns, size exclusion chromatographic columns, immuno-affinity chromatographic columns, heparin sulfate pseudo-affinity chromatographic column, Lectin ligand affinity chromatographic columns and lipofectamine 2000 column columns improves innate immunity. Tumor-specific endogenous siRNA is generated from mutated RNA containing pre-miRNA hairpin through RNA induced silencing complex composed of Dicer, dsRNA binding protein TRBP, and Argo2. Endogenous siRNA is generated by incubating purified RSIC with pre-let-7 hairpin. It is internalized into EVs and T-cells by photochemical methods or using lipofectamine 2000. While undergoing radiation therapy, chemotherapy or surgery, a patient's blood is continuously drawn and processed through the above system. The purified and processed plasma containing EV-exosomes-proteomics-siRNA is returned to the patient. Phototherapy with internalized photosensitive complex-siRNA prevents development of chronic graft versus host disease of adaptive immunotherapy.

As also described under FIG. 25A, the subcellular fractions and the EV-exosome-separated into sucrose gradient cushion are collected from the bottom of the high speed rotating cylindrical rotor 508. In this process, the rotational rpm is reduced to 4,000 and the rotor is brought to a halt without disturbing the sucrose density gradient. After rotor comes to a standstill, flow control valves 518A and 518A2 are closed and air is injected into the rotor through the top of the rotor to push the SDG layers containing subcellular DNA, RNA, extracellular vesicles-exosomes and nanosomes downwards into SDG fraction sample collection tubes 518-I.

Subcellular DNA, RNA, extracellular vesicles-exosomes and nanosomes with higher than 50 S sedimentation coefficient separated into glucose density gradient by continuous flow ultracentrifuge with high speed rotating cylindrical rotor 508 flows through SDG fraction collecting flow line 518C to affinity chromatographic column 518D. SDG fractions are also collected per SDG fraction sample outlet and flow control valve 518H into SDG fraction collection tubes 518-I for biochemical testing and to check integrity of EV-exosomes including testing for CD9, CD63, CD73 and CD90, their size and morphology and for endogenous RNAi, siRNA generation.

The affinity chromatographic columns selected are the same as used for treatment of effluent existing from the top of the rotor described before. They include immobilized Tim4-Fc protein Ca²⁺ magnetic beads affinity column (ITMAC), iZON science's size exclusion chromatographic columns (SEC) qEV, qViro-X or from a series of immobilized metal affinity chromatographic columns (IMAC) or other suitable chromatographic columns selected from the group consisting of other types of SEC, immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. Based on patient specific micro and nano-molecule's separation need, specific chromatographic columns are selected. Such chromatographic columns are routinely used in virus purifications (73).

Tumor-specific endogenous siRNA is also generated from mutated subcellular fragments sedimented in the sucrose gradient. SDG fractions containing mutated subcellular fractions are combined together and used to generate siRNA. Like with the siRNA generating from the effluent existing from the top of the rotor, the tumor specific endogenous siRNA is generated from sucrose density gradient sediment RNA containing pre-miRNA hairpin through RNA induced silencing complex (RISC) composed of Dicer, dsRNA binding protein TRBP, and Argo2 (76). It is generated by incubating purified RSIC with pre-let-7 hairpin (76). This siRNA is then internalized into EVs and into T-cells by electroporation (77) or by photochemical methods or with lipofectamine 2000. Such T-cell treatments silence its evasion from immunity. Chemotherapeutics are also internalized into SDG sediment EVs by electroporation, photochemical methods or with lipofectamine 2000. Labeled EVs containing siRNA and or chemotherapeutics and sucrose gradient plasma flow to sucrose removing cold reservoir 520. SDG fraction collecting flow line 518C leads SDG fraction per fraction to affinity chromatographic column 518D. Chromatographic purified SDG fraction flows into sucrose removing cold reservoir 520 through SDG fraction collecting flow line-2 518C2 where it mixes with effluent plasma existing from the top of the rotor 514. It is cooled to 0° C. to sediment sucrose and the sedimented sucrose 520B collects at the bottom. As with the effluent plasma existing from the top of the rotor 514 the SDG fraction's purified plasma with EV-exosome-siRNA with or without chemotherapeutics is warmed to 370 C with a warming coil 532 in purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A. Alternatively, the purified plasma with EV-exosome-proteomics-siRNA flow valve 525D is opened and the processed plasma with EV-exosome-siRNA and or chemotherapeutics 524 is collected into purified plasma with EV-exosome-proteomics-siRNA collecting bag 525C and preserved for its biochemical analysis and or later administration to the patent. The continuous flow ultracentrifuge and all its accessories are kept in sterile conditions in a sterile environment. The rotor is sterilized online as per manufacturer's instructions. It is also kept sterile and operated in sterile conditions.

FIG. 25C illustrates the same continuous flow ultracentrifuge rotor adapted for plasmapheresis of the pulsed flow apheresis plasma as in FIG. 25A and FIG. 25B but the supernatant exiting from the top hollow driveshaft 510 flows through a series of affinity chromatography columns and nanosomes monitoring with AFM, NTA, DCNA and FCM and the effluent purified supernatant from the chromatographic columns 534 flows back to the high speed rotating cylindrical rotor 508 through its bottom hollow driveshaft 502 and back to the patient or it re-circulates through a series of previously listed chromatography columns namely siRNA affinity columns, Tim4-Fc magnetic beads columns, immobilized Tim4-Fc protein Ca2+ magnetic beads affinity column, iZON science's size exclusion chromatographic columns (SEC) qEV, qViro-X, immobilized metal affinity chromatographic columns, size exclusion chromatographic columns, immuno-affinity chromatographic columns, heparin sulfate pseudo-affinity chromatographic column, lectin ligand affinity chromatographic columns and lipofectamine 2000 column columns that adsorbs and further purifies and separates subcellular nanoparticles, genomics and proteomics in the plasma effluent emerging from the CFUC. The chromatographic columns are selected on the basis of patient specific subcellular fractions present in the plasma effluents. It improves the innate immunity. The flow-line 546 is connected with biochemistry testing devices.

For simultaneous separation of several tumor derived patient specific plasma soluble micro and nano particles including the EVs-exosomes and proteosomes derived from the normal cells and those mutated ones from the tumor cells, the supernatant exiting from the top hollow driveshaft 510 is directed towards several pairs of affinity chromatography columns. In the example illustrated in this FIG. 25C, five pairs of affinity chromatographic columns are shown. They include affinity chromatography column-1, 522A and affinity chromatography column-2 522B; affinity chromatography column-3 522C and affinity chromatography column-4 522D; affinity chromatography column-5 522E and affinity chromatography column-6 522F; affinity chromatography column-7 522G and affinity chromatography column-8 522H: affinity chromatography column-9 522-I and immunoadsorbent affinity chromatography column-10 522J. The five pairs of immunoadsorbent affinity chromatography columns shown here is only an example. The chromatographic columns are selected on the basis of patient specific subcellular fractions present in the plasma effluents. The affinity chromatography columns are interconnected. They are also connected with AFM 536, NTA 538, and DCNA 540 and to FCM 542 for particle tracking. The effluent supernatant exiting from the chromatographic column 522B, 522D, 522F, 522H and 522J flows back to the high speed rotating cylindrical rotor 508 through its bottom hollow driveshaft 502 and back to the patient or the purified processed plasma with EV-exosome-siRNA and sucrose flows to sucrose removing cold reservoir 520 through processed plasma flow line 519. Sucrose removed plasma with EV-exosome siRNA or chemotherapeutics 521 flows through processed plasma flow line 522 to purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A or it is collected into the collecting bag 525C by closing the plasma inlet valve to patient 525B and letting the flow of the purified plasma with EV-exosome and proteomics by opening the purified plasma with EV-exosome-proteomics-siRNA to collecting bag inlet valve 525D. The purified EV-exosome-proteomics-siRNA is collected into purified plasma with EV-exosome-proteomics-siRNA collecting bag 525C. It is transfused back to the patent using the collecting bag's outflow 525E with colleting bag's outflow controlling valve 525F. Alternatively, the processed plasma re-circulates through the affinity chromatographic columns through the supernatant outlet 516 for additional plasma purification. Before the effluent supernatant exiting from the chromatographic columns is injected back into the high speed rotating cylindrical rotor 508, it is cooled to 0° C. with the cooling coil 530. The purified supernatant with EV-exosomes, subcellular particles and proteomics flows into the rotor, out of the rotor and into affinity chromatography columns. The subcellular particles are monitored with AFM, NTA, DCNA and FCM. The plasma flow is controlled with electronic flow direction control switch 544. Before the effluent supernatant exiting from the high speed rotating cylindrical rotor 508 is returned back to patient, it is warmed to w 37° C. with the warming coil 532. Also, before the pulsed flow apheresis plasma is injected into the high speed rotating cylindrical rotor 508 through its bottom hollow driveshaft 502, it is cooled to 0° C. with the cooling coil 530.

The subcellular fractions and the EV-exosome-separated into sucrose gradient cushion are collected from the bottom of the high speed rotating cylindrical rotor 508. In this process, the rotational rpm is reduced to 4,000 and the rotor is brought to a halt without disturbing the sucrose density gradient. After rotor comes to a standstill, flow control valves 518A and 518A2 are closed and air is injected into the rotor through the top of the rotor to push the SDG layers containing subcellular DNA, RNA, extracellular vesicles-exosomes and nanosomes downwards into SDG fraction sample collection tubes 518-I.

Subcellular DNA, RNA, extracellular vesicles-exosomes and nanosomes with higher than 50 S sedimentation coefficient separated into glucose density gradient by continuous flow ultracentrifuge with high speed rotating cylindrical rotor 508 flows through SDG fraction collecting flow line 518C to affinity chromatographic column 518D. SDG fractions are also collected per SDG fraction sample outlet and flow control valve 518H into SDG fraction collection tubes 518-I for biochemical testing and to check integrity of EV-exosomes including testing for CD9, CD63, CD73 and CD90, their size and morphology and for endogenous RNAi, siRNA generation from pooled mutated and normal cellular RNA containing pre-miRNA hairpin through RNA induced silencing complex (RISC) composed of Dicer, dsRNA binding protein TRBP, and Argo2 (76) in SDG fractions.

The affinity chromatographic columns selected for treating both effluent supernatant existing from the top of the rotor and in the sucrose gradient fractions are the same. They were described before. The affinity chromatographic columns for treating the effluent existing from the top of the rotor include affinity chromatography column 1, 522A, 522B affinity chromatography column-2 522B, affinity chromatography column-3 522C, affinity chromatography column-4 522D, affinity chromatography column-5 522E, affinity chromatography column-6 522F, affinity chromatography column-7 522G, affinity chromatography column-8 522H, affinity chromatography column-9 522-I and affinity chromatography column-10 522J. Only one of the affinity chromatographic columns, affinity chromatography column 518D is illustrated for treating the SDG fractions existing from the bottom of the rotor. However like for treatment of the effluent existing from the top of the rotor, a series of affinity columns can be used to treat the sucrose gradient fractions existing from the bottom of the rotor. These affinity columns include immobilized Tim4-Fc protein Ca²⁺ magnetic beads affinity column (ITMAC), iZON science's size exclusion chromatographic columns (SEC) qEV, qViro-X or from a series of immobilized metal affinity chromatographic columns (IMAC) or other suitable chromatographic columns selected from the group consisting of other types of SEC, immuno-affinity chromatographic columns, (IAC), heparin sulfate pseudo-affinity chromatographic column (HAC), Lectin ligand affinity chromatographic columns, (LAC) and lipofectamine 2000 column (LF2000) columns. Based on patient specific micro and nano-molecule's separation need, specific chromatographic columns are selected.

Tumor-specific endogenous siRNA is generated from mutated subcellular RNA containing pre-miRNA hairpin through RNA induced silencing complex (RISC) composed of Dicer, dsRNA binding protein TRBP, and Argo2 in effluent existing from the top of the rotor and SDG fractions existing from bottom of the rotor. SDG fractions containing mutated subcellular fractions are pooled together and used to generate siRNA. Like with the siRNA generating from the effluent existing from the top of the rotor, the tumor specific endogenous siRNA is generated from sucrose density gradient sediment RNA containing pre-miRNA hairpin through RISC composed of Dicer, dsRNA binding protein TRBP, and Argo2 (76). It is generated by incubating purified RSIC with pre-let-7 hairpin (76). This siRNA is then internalized into EVs and into T-cells by electroporation (77) or by photochemical methods or with lipofectamine 2000. Such T-cell treatments silence its evasion from immunity. Chemotherapeutics are also internalized into SDG sediment EVs by electroporation, photochemical methods or with lipofectamine 2000. Chemotherapeutics and siRNA labeled and sucrose gradient plasma flow to sucrose removing cold reservoir 520. SDG fraction collecting flow line 518C leads SDG fraction per fraction to affinity chromatographic column 518D. SDG fraction collecting flow line 570 leads SDG fraction per fraction to the series of affinity chromatographic columns that are connected to effluent processing chromatographic columns, 522A-522J. The electronic flow control valve 572 controls the SDG fraction flow direction either to affinity chromatographic column 518D or to effluent processing affinity chromatographic columns 522A-522J. When effluent from the top of the rotor is processed in chromatographic columns 522A-522J, the electronic flow control valve 572 directing the SDG fraction's flow to these columns is closed to prevent mixing of the SDG gradient fraction with effluent existing from the top of the rotor and entering into chromatographic columns 522A-522J. Chromatographic purified SDG fraction flows into sucrose removing cold reservoir 520 through SDG fraction collecting flow line-2 518C2 where it mixes with effluent plasma existing from the top of the rotor 514. It is cooled to 0° C. to sediment sucrose. The sedimented sucrose 520B collects at the bottom. As with the effluent plasma existing from the top of the rotor 514, the SDG fraction's purified plasma with EV-exosome-siRNA with or without chemotherapeutics is warmed to 37° C. with a warming coil 532 in purified, processed plasma with EV-exosome-siRNA-chemotherapeutics reservoir 523. Purified, processed plasma with EV-exosome-siRNA and or chemotherapeutics 524 is returned to patient through purified, processed plasma with EV-exosome-siRNA-chemotherapeutics inlet to patient 525A. Alternatively, the purified plasma with EV-exosome-proteomics-siRNA flow valve 525D is opened and the processed plasma with EV-exosome-siRNA and or chemotherapeutics 524 is collected into purified plasma with EV-exosome-proteomics-siRNA collecting bag 525C and preserved for its biochemical analysis and or later administration to the patent. The series of affinity chromatography columns are placed in a portable trailer 574 that is kept sterilized in a sterile environment. The continuous flow ultracentrifuge is also kept in sterile condition and in sterile environment. The rotor is sterilized online as per manufacturer's instructions. It is kept sterile and operated in sterile conditions.

The extracorporeal pulse flow apheresis of cell bound proteomics and genomics, combined with molecular apheresis by sucrose density gradient continuous flow ultracentrifugation and affinity chromatography with siRNA affinity columns, Tim4-Fc magnetic beads columns, immobilized Tim4-Fc protein Ca²⁺ magnetic beads affinity column, immobilized metal affinity chromatographic columns, size exclusion chromatographic columns, immuno-affinity chromatographic columns, heparin sulfate pseudo-affinity chromatographic column, lectin ligand affinity chromatographic columns and lipofectamine 2000 columns and treatments with tumor specific endogenous siRNA improves innate immunity. It inhibits tumor recurrence and metastasis. Tumor-specific endogenous siRNA is generated from mutated RNA containing pre-miRNA hairpin through RNA induced silencing complex composed of Dicer, dsRNA binding protein TRBP, and Argo2. Endogenous siRNA is generated by incubating purified RSIC with pre-let-7 hairpin which is internalized into EVs and T-cells and returned to the patient. It silences its evasion from immunity against the tumor. Phototherapy with internalized photosensitive complex-siRNA also prevents development of chronic graft versus host disease of adaptive immunotherapy.

FIG. 25D illustrates a continuous flow ultracentrifuge rotor combined with a series of array rotors adapted for plasmapheresis of the pulsed flow apheresis plasma, its affinity chromatography and online monitoring of the subcellular EV-exosomes, DNA, and RNA-proteomics during the treatment with biochemical testing devices and with AFM, NTA, DCNA and FCM. The array centrifuge disclosed in U.S. Pat. No. 6,387,031 referred before in its entirety (74) is adapted with modifications as methods for additional online purification and separation of subcellular EV-exosomes, DNA, and RNA-proteomics during a patients cancer treatment. In this instance, the EV-exosome-proteomic subcellular components in fractions of pulse flow apheresis and CFUC apheresis effluent plasma are separated simultaneously with rotors ranging from 12 to 96. In FIG. 25D 12 such array rotors are illustrated. The molecular size and weight, the rpm and duration of centrifugation separate them as rotor wall attached precipitates. There may be still remaining few nm and less than a nanometer sized molecules in the supernatant. Thus the supernatant is mostly made free of EV-exosomes and micrometer and nanometer sized mutated proteomic molecules. The remaining submolecular proteomics' separation in array centrifuge is controlled by the higher spinning rpm and duration. They are also further separated and characterized by affinity chromatography consisting of siRNA affinity columns, Tim4-Fc magnetic beads columns, immobilized Tim4-Fc protein Ca²⁺ magnetic beads affinity column, iZON science's size exclusion chromatographic columns (SEC) qEV, qViro-X, immobilized metal affinity chromatographic columns, size exclusion chromatographic columns, immuno-affinity chromatographic columns, heparin sulfate pseudo-affinity chromatographic column, lectin ligand affinity chromatographic columns and lipofectamine 2000 columns. Modified 12 array centrifuges 548 are shown as incorporated with CFUC and affinity chromatographic columns. The array centrifuge 550 shown as adapted for improved methods of separation and characterization of subcellular particles. It consists of titanium rotor for array centrifuge 550 with an upper rotor half 552 and a lower rotor half 554. The array centrifuge rotor shaft 556 is fitted with electronic inlet and outlet flow directing switch 558 for purifying effluent plasma 519B to flow first towards a cooling chamber 521B where sucrose in the purification plasma is sedimented. The sedimented sucrose collects at the bottom of the cooling chamber 521B. The cooled sucrose free plasma 519C flows towards array centrifuges 548. In the original array centrifuge design, a bearing (not shown) presses on to the upper rotor half 552 for speed controlled turning. It is adapted for the controlled rpm for each of the array centrifuges. With variably increasing or decreasing the rpm, the desired cellular submolecules are sedimented onto the polyethylene rotor wall 565 or sedimented at the bottom of the removable and disposable polypropylene rotor insert 564. Alternatively, purification plasma is cooled but sucrose gradient is not precipitated in the cooling chamber 521B and the plasma is centrifuged with sucrose gradient in the array centrifuge. An O-ring 560 absorbs and minimizes the sound generated by the rotation of the rotor. Motors and pulleys that spin the rotors are controlled by a controller (not shown) that is connected to a rotor spin rate controlling computer 562 for computer controlled spinning of the rotors with adjustable speed for each of the rotors. The supernatant is recirculated by reverse flow of the purified plasma to cooling chamber 521B, to cooling chamber 520 and to purified plasma with EV-exosome-proteomics-siRNA collecting bag 525C and back to patient or to chromatographic columns for additional purifications and plasma purification recirculation. The directional arrows 568 indicate the purification plasma's flow directions switched on by the electronic inlet and outlet flow switch 558. Disposable polypropylene rotors 564 are inserted into the titanium rotors. Use of disposable rotors enables patient specific specimen processing without cross contamination. Polypropylene disposable rotors 564 snugly fit into the titanium rotors 550. The advantages of continuous flow ultracentrifugation combined with array centrifuge include sedimentation of biologically active, less than 50S subcellular fractions like 20S, 10S and even 1S purified proteins and nanoparticles that the sucrose gradient continuous flow ultracentrifugation cannot sediment. Less than 50S particles are not sedimented by continuous flow ultracentrifugation (73). It leads many biologically important proteomics and exosomes undetected by continuous flow ultracentrifugation like the 20S mesenchymal cell derived exosome proteosomes (MSC exosomes) (78) 10S functional cytoplasmic exosome (79), cell cycle microtubule stabilizing 15 microtubule associated protein (MAP1S) and the like ones. Ultracentrifugation at 200,000 G for 16.5 hours is used to sediment 20S MSC exosome. Such below 50S exosomes are associated with Alzheimer's disease, Parkinson disease, cardiovascular disease and in prion disease. (78). 20S MSC exosome and circulating 20S proteosomes are capable of reducing misfolded proteins. Hence they significant to treat Alzheimer's disease, Parkinson disease, cardiovascular disease, prion disease and other diseases with unfolded proteins.

The advantages of combined continuous flow ultracentrifugation and ultracentrifugation with array centrifuge include partial separation and purification of less than 50S exosome proteosomes with continuous flow ultracentrifugation at 100,000 G and its additional purification and sedimentation or pelleting with array centrifuge at G-force ranging over 200,000. The rpm of individual rotors in the array centrifuge is adjustable. It is combined with chromatographic additional exosome and proteosomes purification and separation. It is a total analysis of exosomes and proteosomes while the conventional ultracentrifugation based exosomes and proteosomes purification and separation is a partial, incomplete one with discarding valuable subcellular fractions in the supernatants.

The effluent existing from the top of the rotor and the SDG existing from the bottom of the rotor and their affinity chromatography and further treatments and generation of endogenous siRNA and the EV chemotherapeutics preparation for extracorporeal chemotherapy are the same as described in FIG. 25A, FIG. 25B and in FIG. 25C.

FIG. 25E shows a continuous flow zonal rotor than those illustrated in FIG. 25B, FIG. 25C and FIG. 25D and it is combined with a series of array centrifuge and size exclusion chromatographic (SEC) and immuno-affinity chromatographic (IAC) and affinity chromatographic (AC) columns and AFM, NTA, DCNA and FCM for online monitoring of subcellular particles and EV-exosomes during radiation therapy and online extracorporeal chemotherapy.

The cross section of a zonal continuous flow rotor for example, the Beckman continuous flow rotor model CF 32 Ti and JCF-Z is illustrated as an example. The CF 32 Ti rotor runs at maximum 32,000 rpm. It generates 86,100×g at the bottom of the bowl wall and 102,000×g at the inner surface of the bowl wall 586. Likewise, The JCF-Z rotor with standard core runs at maximum 20,000 rpm which generates 32,000×g at the core bottom and 39,000×g at the inner surface of the bowl wall 586. These are low g force for separation of all the subcellular particles and exosomes and proteomics especially those below 50S. Thermo Scientifics' similar rotor, the TCF-32 rotor runs at maximum speed 32,000 rpm that generates maximum 102,000 g which is also insufficient for total separation and pelleting of subcellular particles and exosomes and proteomics especially those below 50S. Even the Thermo-Stovall's alternative CC40 continuous flow ultracentrifuge with cylindrical rotor or other manufacturer's similar continuous flow ultracentrifuges like those from Alpha Wasserman and Hitachi Koki Co., Ltd have only maximum speed of 40,000 rpm which generates maximum 118,000×g. They do not separate or pellet EV exosomes and proteosomes with sedimentation below 50S which is quite unsatisfactory for total EV-exosome analysis and apheresis and for cancer treatment that are described in this invention. The continuous flow ultracentrifugation system combined with array centrifuge with series of rotors that can spin at varying G-Force ranging over 200,000 separates and or pellets the total mutated tumor exosomes and proteosomes and removes them from circulation by their apheresis as described in this invention.

The pulse flow plasma sample flows through plasma injector 528 and enters into continuous flow centrifuge through its central inlet 582. The rotating sealing assembly 580 keeps the fluid lines to remain attached to the rotor during rotation. At low speed rotation, the buffer solution from buffer reservoir 606 or SDG solution reservoir 608 is pumped through buffer line 610 and SDG line 612 by electronically switching on or off the buffer and SDG flow line valve 614. After about a combined volume of about 430 ml buffer and SDG are pumped in at slow rate spinning, the zonal continuous flow rotor 576 is accelerated to operating speed the buffer and SDG flow line valve 614 is switched off and the plasma flow control valve 578 is opened and the pulse flow purified plasma is routed through plasma injector 528 and the central inlet 582 into the bottom of the rotor bowl through the connecting channel to central flow 592. Because of the higher density of the SDG solution, it collects at the periphery of the rotor wall under centrifugal force. At the end of the centrifugation run, rpm is slowly reduced and air is injected through the edge line that makes an airlock at the upper radial channel 584. It prevents disturbance by the dense displacement fluid entering into radial channels 584 during fractionated removal of the SDG layers and disturbing the SDG layers. The dense displacement fluid from the displacement dense fluid reservoir 616 is injected into the rotor through the displacement dense fluid flow line 618 to unload the SDG cushion layers with separated subcellular particles. The rotor contents of SDG fractions flows through the center line to edgeline outlet 594 from where it flows to photometer/flow cell 598 and to SDG fraction collector 600. Based on SDG's density recorded by the photometer/flow cell 598 fractions are collected. It flows to chromatographic affinity columns via electronic flow direction control switch 544. Additional purification of the EV-exosomes and proteosomes and other subcellular contents of the SDG fractions by affinity chromatography and generation of endogenous siRNA and chemotherapeutic-EV for online extracorporeal chemotherapy are the same as those described in FIG. 25A, FIG. 25B and in FIG. 25C.

### Methods of Operation

Methods of near total apheresis of tumor cell derived mutated genomics and proteomics, microsomes, EVs-exosomes, DNA, RNAs, apoptotic bodies, nucleosomes, telomerase, (subcellular particles) and genome silencing with endogenous RNAi-siRNA and miRNA with above described devices including those in FIG. 24, FIG. 25A, FIG. 25B, FIG. 25C, FIG. 25D and FIG. 25E are described below as examples.

1. Methods of Pre and Post Treatment Analysis of Circulating CTC and Subcellular Particles.

Blood is withdrawn from the patient before and after treatments to determine rate of DNA repair and the rate of DNA repair enzymes increase and decrease to normal values after treatments and to determine its relation to abscopal and bystander effects and correlation with its activities in circulating, red cells, granulocytes, macrophages and platelets. Such measurements are repeated daily for 4 days after the treatment and afterwards as needed. In cases of combined radiosurgery and chemotherapy, chemotherapy is administered first and post chemotherapy sample is drawn according to treatment protocols. After radiation therapy a second sample is drawn to assess the combined treatment's effects.

2. Methods of Pulse Flow Cellular and Molecular Aphaeresis Combined with Affinity Chromatography Described in FIG. 24

The CTC, mononuclear white blood cells and platelets carrying tumor specific subcellular particles are removed from circulation first by pulsed flow apheresis combined with affinity chromatography described in FIG. 24 before continuous flow ultracentrifugation apheresis. Two intermittent pulse flow apheresis systems are run simultaneously to have a continuous flow apheresis of the exosomes, microsomes nanosomes including highly increased release of telomerase after chemotherapy/radiosurgery. By 15-30 min gravity sedimentation the RBC, WBC, platelets and plasma are separated. The heavier white blood cells, the red cells and the very bottom circulating tumor cells forms in layers. The plasma with platelets at its bottom collects at the top of the heavier cells. They are separated by gravity differential sedimentation as described in FIG. 24. A series of affinity columns and a series of microfilters separate and remove the CTC, mononuclear white cells and platelets and parts of subcellular fractions and enzymes, telomerase from the plasma. Rapid flow cytometry of the cells sampled by pulse apheresis after chemotherapy/radiosurgery is used to monitor gamma H2AX containing cells as a measure of removal of CTC (82) and subcellular particles. The blood components are also passed thorough affinity chromatograms. Heparin mimics as a DNA binding polyanionic structure nucleic acid (83). Disposable DNA binding proteins with HiTrap heparin column or cellulose activated charcoal coated with heparin is also used as affinity chromatograms. The supernatant plasma filtered through the filters in FIG. 24A, 392, 429, 406,478PL, 424,476W, 4- and 478R is routed through the continuous flow centrifuge while their cellular elements collected in the collection bags are preserved for future use or returned to patient after removing the cell and cell membrane bound EV-exosomes containing tumor cell derived subcellular particles. The circulating cells, the red cells, granulocytes, monocytes, lymphocytes, macrophages and the platelets harvested and purified by pulse flow apheresis is further treated in hypoxic conditions to release its EVs-exosomes with subcellular particles (84) It is then washed with acidified PBS to remove both free and cell membrane bound exosomes and subcellular particles (85). They are preserved for future use or treated by extracorporeal immunotherapy to neutralize any remaining tumor cell derived exosomes. Such treated circulating cells are returned to the patient as part of combined innate and adaptive immunotherapy directed against tumor cell derived EVs-exosomes bound to circulating the red cells, granulocytes, monocytes, lymphocytes, macrophages and the platelets.

3. Methods of Continuous Flow Ultracentrifugation Molecular Aphaeresis Combined with Affinity Chromatography Described in FIGS. 25B, 25C, 25D and 25E

Removal of plasma soluble cell debris, larger micro and nano particles, cell membranes, normal cell and tumor cell derived proteins, and subcellular particles including apoptotic bodies, DNA and RNAs, microsomes, exosomes and nanosomes, telomere and telomerase, ATM and ATM kinase after pulse flow apheresis by pulse flow apheresis system 378 is directed into the continuous flow ultracentrifuge systems illustrated in FIG. 25B, FIG. 25C FIG. 25D and FIG. 25E. Subcellular DNA, RNA, extracellular vesicles-exosomes and nanosomes with higher than 50 S sedimentation coefficient is separated into sucrose density gradient by continuous flow ultracentrifuge with high speed rotating rotor 508 as shown in FIG. 25B, FIG. 25C and FIG. 25D. Additional subcellular DNA, RNA, extracellular vesicles-exosomes and nanosomes with lower than 50 S sedimentation coefficient is separated into sucrose density gradient by the combined continuous flow ultracentrifuge with high speed rotating rotor 508 and with array centrifuge 548 as illustrated in FIG. 25D or with zonal continuous flow rotor 576 and array centrifuge 548 as illustrated in FIG. 25E.

4. After Radiosurgery, Separation of Cancer and Cancer Stem Cell Derived Exosomes from Exosomes Derived from Normal Cells at 24 and or 48 h

In response to cancer treatments, especially after local radiation therapy, large burst of cellular fragments including micro and nano particles, cell membranes, normal cell and tumor cell derived proteins, and subcellular particles including apoptotic bodies, DNA and RNAs, microsomes, exosomes and nanosomes, telomere and telomerase, ATM and ATM kinase are released into circulation. Its high peak occurs within 24 to 48 hours. Identification of the phenotypic variation among the tumor derived exosomes by various methods during the window of this high peak periods is described below as examples.

The SDG and SEC fractions are analyzed for patient specific cancer and cancer stem cell phenotype. The generally used genomics and proteomic testing is adapted for such analysis. They include:
1. Oncosome immunohistochemistry (35),
2. In-vitro gene silencing, Immunoblotting and siRNA stability assay (63),
3. size-exclusion chromatography, transmission EM, RNA isolation, qRT-PCR Assays, miRNA profiling, miRNA profiling data analysis, immunoprecipitation and immunoblotting, and data analysis for miRNA in plasma fraction Ago2 immunoprecipitates (22),
4. Inducible RAD51 assay, indirect immunofluorescence RAD51 staining, confocal microscope image acquisition, gamma H2AX foci counting, Western blotting, subcellular fractionation ssDNA and dsDNA quantification, RNA microarray (64),
5. Microarray mRNA analysis, microarray miRNA analysis, real-time polymerase chain reaction (PCR), miRNA transfection with Lipofectamine 2000, 3′UTR Reporter assay, in vitro translation, flow cytometry 34,
6. Analysis of exosomal RNA content by sequencing, tracing exosomal uptake in the in vitro system (31).
7. A large-scale targeted proteomics assay resource based on an in vitro human proteome (81)
8. EV-Exosomes derived from cancer cells and undifferentiated cancer cells and their phenotyping:
   a. AFM measurements of shape, height, width, surface roughness and stiffness combined with fluorescence microscopy of gold nanoparticles coated with antibody against undifferentiated cancer stem cell's antigens and which is bound to normal cells and to exosome and exosome proteins, its antigens, DNAs and RNAs and its comparison with antigen-antibody binding differentiated cancer cell's antigens.
   b. AFM measurements of shape, height, width, surface roughness and stiffness of exosomes combined with fluorescence microscopy of exosomes bound to normal cells and to undifferentiated cancer stem cell specific antigens selected from the list of cancer stem cell antigens and they have mutated CTCF with different height and length and DNA looping and its comparison with antigen-antibody binding for the differentiated cancer cell's antigens.
   c. AFM measurements of shape, height, width, surface roughness and stiffness of exosomes combined fluorescence microscopy of exosomes bound to normal cells and to undifferentiated cancer stem cell specific antigens and they have cancer treatment resistance like resistance to 5-flurouracil (5-FU) when cytosine deaminase-uracil phosphoribosyl transferase (CD-UPRT) fusion gene is present and its comparison with antigen-antibody binding for differentiated cancer cell's antigens.
   d. AFM combined fluorescence microscopy of exosomes bound to normal cells and to undifferentiated cancer stem cell specific antigens showing different height and width, surface roughness and stiffness histograms in the purified exosome DNA and their double strand break and homologues DNA repair deficiency after cancer radiosurgery and chemotherapy and its comparison with antigen-antibody binding for the differentiated cancer cell antigens.
   e. AFM combined fluorescence microscopy of exosomes bound to normal cells and to undifferentiated cancer stem cell specific antigens showing different shape, height and width surface roughness and stiffness exosome in response to radiation therapy and chemotherapy and its comparison with antigen-antibody binding for differentiated cancer cell's antigens.
   f. AFM combined fluorescence microscopy of exosomes bound to normal cells and to undifferentiated cancer stem cell specific antigens selected from the list of cancer stem cell antigens and the cancer stem cell exosome's poly (ADP) ribose polymerase (PARP) cleavage in response to radiation therapy and chemotherapy and its associated changes in cancer stem cell's shape, height and width surface roughness and stiffness and its comparison with antigen-antibody binding for differentiated cancer cell's antigens.
   g. AFM combined fluorescence microscopy of exosomes bound to normal cells and to undifferentiated cancer stem cell specific antigens selected and the presence of Rad50/MRE11/NBS1 (MRN Complex) in ER, PR and HER2 negative breast cancer patient's exosomes with changes in their shape, height and width surface roughness and stiffness and its comparison with antigen-antibody binding to differentiated cancer cell's antigens.
   h. AFM combined fluorescence microscopy of exosomes bound to normal cells and to undifferentiated cancer stem cell specific antigens and the presence of Warburg glycolytic glutamate in exosomes with associated changes in their shape, height, width, surface roughness and stiffness and its comparison with antigen-antibody binding to differentiated cancer cell's antigens.
   i. AFM combined fluorescence microscopy of exosomes bound to normal cells and to undifferentiated cancer stem cell specific antigens and the cancer stem cell exosomes without greatly diminished caspase activity and its associated changes in exosomes shape, height and width, surface roughness and stiffness in comparison with antigen-antibody binding of differentiated cancer cell's antigens.
9. Tumor Cell's and Normal Cell's Exosome Analysis by Disc Centrifuge and by Nanoparticle Tracking Analysis (NTA)
   A quick analysis of the fraction with peak exosome content and its size and shape ranging from 10 nm to 100 nm is determined with a disc centrifuge and the exosome imaging with AFM. This exosome preparation contains both exosomes from cancer and cancer stem cells and those from the normal cells.
   Its aliquot is suspended in the PBS and its size, shape and movements are recorded with NTA software. The video images captured by the NTA automatically and simultaneously record thousands of exosome's locations, their movements and centre of each and every particle and measures the average distance it moves per frame. This information on the characteristics of the exosome like the size, relative intensity and concentration in 2-D and 3-D formats is displayed.
10. Comparative AFM/NTM/SDNA Phenotypic Analysis of Cancer Stem Cell Exosomes and Normal Tissue Exosomes in SDG Fractions Containing Purified Total Exosomes (PTE)
   For the purpose of assessing the exosome in the SDG fractions to determine if they are derived from undifferentiated cancer stem cells, differentiated cancer cells or normal cells, the following guidelines are followed.
   First, the exosomes in an aliquot of SDGUF are tested for undifferentiated cancer stem cell antigens and their specific antibody binding.
   Second, the remaining exosomes in the same aliquot of SDGUF are tested for known, differentiated cancer cell markers.
   If both the first and second group's testing for the antigen antibody binding of exosomes shows their respective antigen-antibody specificity, then those exosomes and subcellular particles and are marked as derived from undifferentiated cancer stem cells. It is one type of undifferentiated cancer stem cell exosome's phenotype.
   If the exosomes have only undifferentiated cancer stem cell antigen-antibody binding, and no or poor binding to generally known cancer antigen markers, then such subcellular particles and exosomes are marked as derived from undifferentiated cancer stem cells but of a different phenotype.
   If there are no undifferentiated cancer stem cell antigen-antibody binding but has binding only to generally known cancer cell markers, then such subcellular particles and exosomes are marked as derived from more differentiated cancer cells.
   The exosomes with poor or no cancer cell antigen-antibody binding are marked as derived from normal tissue. They are the remaining exosome in the same aliquot SDGUF after separation of the exosomes derived from undifferentiated cancer stem cells and those exosomes from differentiated cancer cells.
   Thus the characteristics in exosomes derived from the undifferentiated cancer stem cells and differentiated cancer cells and their correlation with presence or absence of generally known cancer cell markers will indicate the predominant phenotype of a tumor from which the tested cancer cell exosome have originated.

The disclosures of all references cited herein are hereby incorporated as references. Listing of references herein is not intended to be a representation that a complete search of all relevant art has been made, or that no more pertinent art than that listed exists, or that the listed art is material to patentability. Nor should any such representation be inferred.

While this inventor has described what the prescribed embodiments of the present invention are presently, other and further changes and modifications could be made without departing from the scope of the invention and it is intended by this inventor to claim all such changes and modifications. Accordingly, it should be also understood that the present disclosure has been presented for purposes of example rather than limitation, and does not preclude inclusion of such modifications, variations and/or additions to the present subject matter as would be readily apparent to one of ordinary skill in the art.

EV-Patent Application: Table of Contents

| No | Title | Page |
|---|---|---|
| 1 | CIP-Information | 1 |
| 2 | Background | 1 |
| 3 | EVs and Intercellular communication | 4 |
| 4 | Cellular and organ targeting by EVs | 4 |
| 5 | Circulating RNAs | 5 |
| 6 | Pulse flow cellular apheresis | 8 |
| 7 | Continuous flow cell separator | 8 |
| 8 | Continuous flow ultracentrifugation plasmapheresis of | 9 |
| | circulating tumor derived EVs | |
| 9 | Prior art affinity chromatography with lectins | 10 |
| 10 | EVs and exRNA associated diseases | 11 |
| 12 | Lymph node metastasis and circulating EVs and exRNAs | 13 |
| 13 | Large oncosome and metastasis | 14 |
| 14 | Extracellular RNA (exRNA) | 15 |
| 15 | Cancer stem cell EVs and exRNAs and cancer treatment | 16 |
| 16 | MicroRNAs (miRNAs) | 18 |
| 17 | Table 1, Poor prognostic cancer associated miRNAs | 18 |
| 18 | DNA Damage Response (DDR) | 24 |
| 19 | Table 2, Potential metastasis causing mutated DNA | 24 |
| | Damage Repair gens | |
| 20 | Extracorporeal Chemotherapy aided by Pulse-Flow- | 26 |
| | Continuous-Flow Ultracentrifugation and Chromatography | |
| 21 | Metastasis inhibiting Radiotherapy's AGO2-RISC- | 27 |
| | RAD51-diRNA-RNAi-miRNA-siRNA enhanced | |
| | Immunotherapy and Extracorporeal Chemotherapy | |
| 22 | Brief summary of the invention | 28 |
| 23 | Brief description of the Drawings | |
| 24 | Reference Numerals | 29 |
| 25 | Detailed Description of the Preferred Embodiments | 33 |
| 26 | FIG. 24 | 33 |
| 27 | FIG. 25A | |
| 28 | FIG. 25B | |
| 29 | FIG. 25C | |
| 30 | Selected in-vitro and in-vivo methods of EVs-exosome, | 42 |
| | RNA, DNA cellular interaction | |

What is claimed:

1. A device for circulating cell and subcellular nanoparticle's separation and removal comprising:
a. apparatus for pulse flow aphaeresis of red cells, white cells, platelets and tumor cells and plasmapheresis and filtration of said components in blood;
b. pulse flow aphaeretic system attached to affinity chromatograms;
c. pulse flow aphaeretic system combined with microfilters;
d. pulse flow apheresis system attached to immune affinity columns;
e. pulse flow apheresis system attached to a continuous flow ultracentrifuge rotor;
f. a continuous flow ultracentrifuge for plasma soluble molecule's apheresis;
g. a continuous flow ultracentrifuge rotor connected to a series of affinity columns;
h. a continuous flow ultracentrifuge rotor connected to size exclusion chromatography columns;
i. a continuous flow ultracentrifuge rotor connected to iZON science's modified size exclusion chromatography columns;
j. a continuous flow ultracentrifuge rotor connected to immobilized Tim4-Fc protein Ca²⁺ magnetic beads affinity columns;
k. a continuous flow ultracentrifuge rotor connected to immobilized metal affinity chromatography columns
l. a continuous flow ultracentrifuge rotor connected to immuno-affinity chromatography columns;
m. a continuous flow ultracentrifuge rotor connected to heparin sulfate pseudo-affinity chromatography columns;
n. a continuous flow ultracentrifuge rotor connected to lectin ligand affinity chromatography columns;
o. a continuous flow ultracentrifuge rotor connected to lipofectamine 2000 chromatography columns;
p. a continuous flow ultracentrifuge rotor and affinity chromatography columns connected to array ultracentrifuge with rotors ranging from 12-96;
q. a continuous flow ultracentrifuge rotor and affinity chromatography columns connected to an array ultracentrifuge with rotors ranging from 12-96 and each said rotors spins at adjustable rpm;
r. array centrifuge rotors capable of spinning at adjustable g-force ranging from 100,000 to 200,000;
s. array centrifuge rotor's spin rate controlling computer;
t. a pulse flow apheresis system, a continuous flow ultracentrifuge rotor and affinity chromatography columns connected to array ultracentrifuge rotors and to processed plasma collecting and cooling chambers;
u. a pulse flow apheresis system, a continuous flow ultracentrifuge rotor and affinity chromatography columns connected to array ultracentrifuge rotors and to processed plasma cooling and sucrose precipitating chambers;
v. a pulse flow apheresis system, a continuous flow ultracentrifuge rotor and affinity chromatography columns connected to processed plasma collecting and cooling chambers;
w. a pulse flow apheresis system, a continuous flow ultracentrifuge rotor, affinity chromatography columns and array centrifuge with a series of rotors connected to processed plasma cooling and sucrose precipitating chambers;
x. a pulse flow apheresis system, a continuous flow ultracentrifuge rotor, affinity chromatography columns connected to flow cytometer (FCM), atomic force microscope (AFM), nanoparticle tracking analysis (NTA) system, disc centrifuge nanoparticle analysis (DCNA) system and to large-scale targeted proteomics assay resource;
y. a pulse flow apheresis system, a continuous flow ultracentrifuge rotor, affinity chromatography columns and array centrifuge with a series of array centrifuge rotors connected to flow cytometer, atomic force microscope, nanoparticle tracking analysis (NTA) system, disc centrifuge nanoparticle analysis (DCNA) system and to large-scale targeted proteomics assay resource;
z. a pulse flow apheresis system, a continuous flow ultracentrifuge rotor, affinity chromatography columns connected to sucrose density gradient fraction collector;
aa. a pulse flow apheresis system, a continuous flow ultracentrifuge rotor, affinity chromatography columns and an array centrifuge with a series of rotors connected to photometer/flow cell and to sucrose density gradient fraction collector;
bb. blood and red blood cells, leucocytes, lymphocytes, platelets and plasma collection bags attachable to pulse flow combined ultracentrifuge apheresis system.

2. Methods of apheresis of circulating cells and plasma soluble subcellular particles, extracellular vesicles, exosomes, proteomics and genomics and therapeutic applications of said endogenous subcellular particles comprising the steps of:
a. therapeutic apheresis of circulating mutated cellular and subcellular particles to minimize metastasis and tumor recurrence;
b. therapeutic apheresis of large burst of mutated cellular and subcellular particles, extracellular vesicles and exosomes released into circulation in response to cancer treatments to inhibit abscopal metastasis in distant organs;
c. therapeutic apheresis of large burst of mutated cellular and subcellular particles, extracellular vesicles and exosomes released into circulation in response to radiation therapy to inhibit abscopal metastasis in distant organs;
d. therapeutic apheresis of large burst of mutated cellular and subcellular particles, extracellular vesicles and exosomes released into tumor environment in response to radiation therapy to inhibit metastasis from bystander effect;
e. therapeutic apheresis of large burst of mutated cellular and subcellular particles, extracellular vesicles and exosomes released into circulation in response to cancer treatments to inhibit platelet activation and abscopal metastasis in distant organs;
f. therapeutic apheresis of large burst of mutated cellular and subcellular particles, extracellular vesicles and exosomes released into circulation in response to cancer treatments to inhibit macrophage activation into tumor promoting M2-like macrophage and abscopal metastasis in distant organs;
g. therapeutic apheresis of large burst of mutated cellular and subcellular particles, extracellular vesicles and exosomes released into circulation in response to cancer treatments that inhibit T-lymphocytes, macrophage, platelets and innate immunity against tumor;
h. therapeutic apheresis of large burst of mutated cellular and subcellular particles, extracellular vesicles and exosomes released into circulation in response to cancer treatments that cause T-lymphocyte's escape from innate and adaptive immune response to tumor;
i. therapeutic apheresis of large burst of mutated cellular and subcellular particles, extracellular vesicles and exosomes released into circulation in response to radiation therapy to overcome therapeutic escape due to proteomic changes in extracellular vesicles and exosomes caused by radiation;
j. mutated genome silencing with endogenous RNAi-siRNA generated from DNA damage repair response releasing Ago-2-RISC-Rad-51-diRNA-RNAi-miRNA complexes after radiation therapy and cancer treatments;
k. mutated genome silencing with endogenous RNAi-siRNA generated from DNA damage repair response releasing Ago-2-RISC-Rad-51-diRNA-RNAi-miRNA complexes separated by continuous flow ultracentrifugation and siRNA and RNAi precipitation onto array centrifuge rotors and administration such prepared siRNA and RNAi back to patient;
l. mutated genome silencing with endogenous RNAi-siRNA generated from DNA damage repair response releasing Ago-2-RISC-Rad-51-diRNA-RNAi-miRNA complexes captured onto affinity columns and RNAi, siRNA elution and administration back to patient;
m. mutated genome silencing with endogenous siRNA generated by incubating purified RSIC with pre-let-7 hairpin;
n. bonding siRNA with tumor cell derived extracellular vesicles by electroporation for cell silencing;
o. bonding siRNA with tumor cell derived extracellular vesicles by photochemical methods for cell silencing;
p. bonding siRNA with tumor cell derived extracellular vesicles with lipofectamine 2000 for cell silencing;
q. bonding siRNA with T-lymphocytes by electroporation to inhibit T-cell evasion from immunity;
r. bonding siRNA with T-lymphocytes by photochemical method to inhibit T-lymphocyte's evasion from immunity;
s. bonding siRNA with T-lymphocytes with lipofectamine 2000 to inhibit T-lymphocyte's evasion from immunity;
t. inhibition of chronic graft versus host disease with tumor cell derived extracellular vesicles' internalized photosensitive complex-siRNA;
u. inhibition of chronic graft versus host disease with T-lymphocytes with internalized photosensitive complex-siRNA;
v. internalization of chemotherapeutics into tumor cell's extracellular vesicles and exosomes purified by continuous flow ultracentrifuge and array ultracentrifuge by electroporation for extracorporeal chemotherapy;
w. internalization of chemotherapeutics into tumor cell's extracellular vesicles and exosomes purified by continuous flow ultracentrifuge and array ultracentrifuge by photochemical methods for extracorporeal chemotherapy;
x. internalization of chemotherapeutics into tumor cell's extracellular vesicles and exosomes purified by continuous flow ultracentrifuge and array ultracentrifuge with lipofectamine 2000 for extracorporeal chemotherapy;
y. tumor seeking extracorporeal chemotherapy with tumor cell's extracellular vesicles with internalized chemotherapeutics;
z. combined online radiation therapy, surgery, chemotherapy and therapeutic apheresis of large burst of mutated cellular and subcellular particles, extracellular vesicles and exosomes released into circulation in response to such treatments with combined pulse flow apheresis and continuous flow ultracentrifuge and array centrifuge ultracentrifugation apheresis;
aa. combined online radiation therapy and chemotherapy and therapeutic apheresis of large burst of mutated cellular and subcellular particles, extracellular vesicles and exosomes released into circulation in response to such treatments with combined pulse flow apheresis and continuous flow ultracentrifuge and array centrifuge ultracentrifugation apheresis;
bb. near total apheresis of mutated cellular and subcellular particles, extracellular vesicles and exosomes released into circulation in response to cancer treatments by apheresis of the entire circulating blood and plasma several times during a treatment cycle lasting several hours.
cc. therapeutic apheresis of large burst of mutated cellular and subcellular particles, extracellular vesicles and exosomes released into circulation in response to cancer chemotherapy to inhibit abscopal metastasis in distant organs;
dd. apheresis of mutated extracellular vesicles carrying apoptotic bodies, microsomes, exosomes, oncosomes, DNA and DNA fragments and microRNAs;
ee. apheresis of circulating plasma soluble mutated subcellular particles extracellular vesicles, exosomes, proteosomes to inhibit early niche metastatic process;
ff. apheresis of extracellular vesicles carrying vascular endothelial growth factor to inhibit tumor vascular formation;
gg. apheresis of early metastatic lymph node seeding of extracellular vesicles and exosomes;
hh. apheresis of benign metastatic lymphangioleiomyomatosis (LAM) causing extracellular vesicles;
ii. apheresis of extracellular vesicles and exosomes traveling to sentinel lymph nodes and causing metastatic melanomas;
jj. apheresis of extracellular vesicles and exosomes causing metastatic melanomas;
kk. pulse flow aphaeresis of red cells, white cells, platelets and tumor cells and plasmapheresis and filtration of said components in blood;
ll. pulse flow aphaeresis plasma's filtration with microfilters;
mm. pulse flow aphaeresis filtered plasma's size exclusion chromatography with chromatographic columns;
nn. chromatography of pulse flow aphaeresis' filtered plasma with immune affinity columns;
oo. removal of subcellular particle's dissolved in pulse flow apheresis plasma with a continuous flow ultracentrifuge rotor connected to pulse flow apheresis system;
pp. a continuous flow plasma ultracentrifugation apheresis, removal and characterization of plasma soluble subcellular particles, extracellular vesicles, exosomes, proteosomes and genomes with higher than 50S sedimentation coefficient in sucrose density gradient;
qq. a continuous flow plasma ultracentrifugation aphaeresis for removal and characterization of plasma soluble mutated molecular subcellular particles, extracellular vesicles, exosomes, proteosomes and genomes with higher than 50S sedimentation coefficient in sucrose density gradient;
rr. a continuous flow ultracentrifugation rotor and a series of array centrifuge rotors combined plasma ultracentrifugation for removal and characterization of plasma soluble subcellular particles, extracellular vesicles, exosomes, proteosomes and genomes with lower than 50S sedimentation coefficient in sucrose density gradient;
ss. a continuous flow ultracentrifugation rotor and a series of array centrifuge rotors combined plasma ultracentrifugation for removal and characterization of mutated plasma soluble molecular subcellular particles, extracellular vesicles, exosomes, proteosomes and genomes with lower than 50S sedimentation coefficient in sucrose density gradient;
tt. a continuous flow ultracentrifugation rotor combined with a series of array centrifuge rotors with adjustable rpm and g-force for plasma ultracentrifugation for removal and characterization of plasma soluble subcellular particles, extracellular vesicles, exosomes, proteosomes and genomes with lower than 50S sedimentation coefficient in sucrose density gradient;
uu. a continuous flow ultracentrifugation rotor combined with a series of array centrifuge rotors for plasma ultracentrifugation for removal and characterization of mutated plasma soluble molecular subcellular particles, extracellular vesicles, exosomes, proteosomes and genomes with lower than 50S sedimentation coefficient in sucrose density gradient;
vv. molecular sieve separation and characterization of plasma soluble molecular subcellular particles with higher than 50S sedimentation coefficient with a continuous flow ultracentrifuge rotor connected to a series of affinity columns;
ww. molecular sieve separation and characterization of plasma soluble molecular subcellular particles with higher than 50S sedimentation coefficient with a continuous flow ultracentrifuge rotor connected to size exclusion chromatography columns;
xx. molecular sieve separation and characterization of plasma soluble molecular subcellular particles with higher than 50S sedimentation coefficient with a continuous flow ultracentrifuge rotor connected to iZON science's modified size exclusion chromatography columns;
yy. molecular sieve separation and characterization of plasma soluble molecular subcellular particles with higher than 50S sedimentation coefficient with a continuous flow ultracentrifuge rotor connected to immobilized Tim4-Fc protein Ca²⁺ magnetic beads affinity columns;
zz. molecular sieve separation and characterization of plasma soluble molecular subcellular particles with higher than 50S sedimentation coefficient with a continuous flow ultracentrifuge rotor connected to immobilized metal affinity chromatographic columns;
aaa. molecular sieve separation and characterization of plasma soluble molecular subcellular particles with higher than 50S sedimentation coefficient with a continuous flow ultracentrifuge rotor connected to immuno-affinity chromatographic columns;
bbb. molecular sieve separation and characterization of plasma soluble molecular subcellular particles with higher than 50S sedimentation coefficient with a continuous flow ultracentrifuge rotor connected to heparin sulfate pseudo-affinity chromatography columns;
ccc. molecular sieve separation and characterization of plasma soluble molecular subcellular particles with higher than 50S sedimentation coefficient with a continuous flow ultracentrifuge rotor connected to lectin ligand affinity chromatography columns;
ddd. molecular sieve separation and characterization of plasma soluble molecular subcellular particles with higher than 50S sedimentation coefficient with a continuous flow ultracentrifuge rotor connected to lipofectamine 2000 chromatography columns;
eee. molecular sieve separation and characterization of plasma soluble mutated molecular subcellular particles with higher than 50S sedimentation coefficient with a continuous flow ultracentrifuge rotor connected to a series of affinity columns;
fff. molecular sieve separation and characterization of plasma soluble mutated molecular subcellular particles with higher than 50S sedimentation coefficient with a continuous flow ultracentrifuge rotor connected to size exclusion chromatography columns;
ggg. molecular sieve separation and characterization of plasma soluble mutated molecular subcellular particles with higher than 50S sedimentation coefficient with a continuous flow ultracentrifuge rotor connected to iZON science's modified size exclusion chromatography columns;
hhh. molecular sieve separation and characterization of plasma soluble mutated molecular subcellular particles with higher than 50S sedimentation coefficient with a continuous flow ultracentrifuge rotor connected to immobilized Tim4-Fc protein Ca²⁺ magnetic beads affinity columns;
iii. molecular sieve separation and characterization of plasma soluble mutated molecular subcellular particles with higher than 50S sedimentation coefficient with a continuous flow ultracentrifuge rotor connected to immobilized metal affinity chromatographic columns;
jjj. molecular sieve separation and characterization of plasma soluble mutated molecular subcellular particles with higher than 50S sedimentation coefficient with a continuous flow ultracentrifuge rotor connected to immuno-affinity chromatographic columns;
kkk molecular sieve separation and characterization of plasma soluble mutated molecular subcellular particles with higher than 50S sedimentation coefficient with a continuous flow ultracentrifuge rotor connected to heparin sulfate pseudo-affinity chromatography columns;
lll. molecular sieve separation and characterization of plasma soluble mutated molecular subcellular particles with higher than 50S sedimentation coefficient with a continuous flow ultracentrifuge rotor connected to lectin ligand affinity chromatography columns;
mmm. molecular sieve separation and characterization of plasma soluble mutated molecular subcellular particles with higher than 50S sedimentation coefficient with a continuous flow ultracentrifuge rotor connected to lipofectamine 2000 chromatography columns;
nnn. molecular sieve separation and characterization of plasma soluble molecular subcellular particles with lower than 50S sedimentation coefficient with a continuous flow ultracentrifuge rotor and affinity chromatography columns connected to a continuous flow array ultracentrifuge having an array of rotors ranging from 12-96 and having rotors with adjustable rpm and g-force ranging from 100,000 to 200,000;
ooo. molecular sieve separation and characterization of plasma soluble molecular subcellular particles with lower than 50S sedimentation coefficient with a continuous flow ultracentrifuge rotor and size exclusion chromatography columns connected to a continuous flow array ultracentrifuge having an array of rotors ranging from 12-96 and having rotors with adjustable rpm and g-force ranging from 100,000 to 200,000;
ppp. molecular sieve separation and characterization of plasma soluble molecular subcellular particles with lower than 50S sedimentation coefficient with a continuous flow ultracentrifuge rotor and iZON science's modified size exclusion chromatography columns connected to a continuous flow array ultracentrifuge having an array of rotors ranging from 12-96 and having rotors with adjustable rpm and g-force ranging from 100,000 to 200,000;
qqq. molecular sieve separation and characterization of plasma soluble molecular subcellular particles with lower than 50S sedimentation coefficient with a continuous flow ultracentrifuge rotor and immobilized Tim4-Fc protein Ca2+ magnetic beads affinity columns connected to a continuous flow array ultracentrifuge having an array of rotors ranging from 12-96 and having rotors with adjustable rpm and g-force ranging from 100,000 to 200,000;
rrr. molecular sieve separation and characterization of plasma soluble molecular subcellular particles with lower than 50S sedimentation coefficient with a continuous flow ultracentrifuge rotor and immuno-affinity chromatographic columns connected to a continuous flow array ultracentrifuge having an array of rotors ranging from 12-96 and having rotors with adjustable rpm and g-force ranging from 100,000 to 200,000;
sss. molecular sieve separation and characterization of plasma soluble molecular subcellular particles with lower than 50S sedimentation coefficient with a continuous flow ultracentrifuge rotor and heparin sulfate pseudo-affinity chromatography columns connected to a continuous flow array ultracentrifuge having an array of rotors ranging from 12-96 and having rotors with adjustable rpm and g-force ranging from 100,000 to 200,000;
ttt. molecular sieve separation and characterization of plasma soluble molecular subcellular particles with lower than 50S sedimentation coefficient with a continuous flow ultracentrifuge rotor and lectin ligand affinity chromatography columns connected to a continuous flow array ultracentrifuge having an array of rotors ranging from 12-96 and having rotors with adjustable rpm and g-force ranging from 100,000 to 200,000;
uuu. molecular sieve separation and characterization of plasma soluble molecular subcellular particles with lower than 50S sedimentation coefficient with a continuous flow ultracentrifuge rotor and lipofectamine 2000 chromatography columns connected to a continuous flow array ultracentrifuge having an array of rotors ranging from 12-96 and having rotors with adjustable rpm and g-force ranging from 100,000 to 200,000;
vvv. molecular sieve separation and characterization of plasma soluble mutated molecular subcellular particles with lower than 50S sedimentation coefficient with a continuous flow ultracentrifuge rotor and size exclusion chromatography columns connected to a continuous flow array ultracentrifuge having an array of rotors ranging from 12-96 and having rotors with adjustable rpm and g-force ranging from 100,000 to 200,000;
www. molecular sieve separation and characterization of plasma soluble mutated molecular subcellular particles with lower than 50S sedimentation coefficient with a continuous flow ultracentrifuge rotor and iZON science's modified size exclusion chromatography columns connected to a continuous flow array ultracentrifuge having an array of rotors ranging from 12-96 and having rotors with adjustable rpm and g-force ranging from 100,000 to 200,000;
xxx. molecular sieve separation and characterization of plasma soluble mutated molecular subcellular particles with lower than 50S sedimentation coefficient with a continuous flow ultracentrifuge rotor and immobilized Tim4-Fc protein Ca2+ magnetic beads affinity columns connected to a continuous flow array ultracentrifuge having an array of rotors ranging from 12-96 and having rotors with adjustable rpm and g-force ranging from 100,000 to 200,000;
yyy. molecular sieve separation and characterization of plasma soluble mutated molecular subcellular particles with lower than 50S sedimentation coefficient with a continuous flow ultracentrifuge rotor and immuno-affinity chromatographic columns connected to a continuous flow array ultracentrifuge having an array of rotors ranging from 12-96 and having rotors with adjustable rpm and g-force ranging from 100,000 to 200,000;
zzz. molecular sieve separation and characterization of plasma soluble mutated molecular subcellular particles with lower than 50S sedimentation coefficient with a continuous flow ultracentrifuge rotor and heparin sulfate pseudo-affinity chromatography columns connected to a continuous flow array ultracentrifuge having an array of rotors ranging from 12-96 and having rotors with adjustable rpm and g-force; ranging from 100,000 to 200,000
aaaa. molecular sieve separation and characterization of plasma soluble mutated molecular subcellular particles with lower than 50S sedimentation coefficient with a continuous flow ultracentrifuge rotor and lectin ligand affinity chromatography columns connected to a continuous flow array ultracentrifuge having an array of rotors ranging from 12-96 and having rotors with adjustable rpm and g-force ranging from 100,000 to 200,000;
bbbb. molecular sieve separation and characterization of plasma soluble mutated molecular subcellular particles with lower than 50S sedimentation coefficient with a continuous flow ultracentrifuge rotor and lipofectamine 2000 chromatography columns connected to a continuous flow array ultracentrifuge having an array of rotors ranging from 12-96 and having rotors with adjustable rpm and g-force ranging from 100,000 to 200,000;
cccc. computer and computer software aided control of array centrifuge rotor's varying spin rate that separates subcellular particles based on their molecular weights and configurations in gradient solutions;
dddd. separation of high density sucrose from sucrose density gradient by cold precipitation before administration of the treated aphaeretic plasma back to patient;
eeee. aphaeretic processed plasma collection to sterile blood and blood component's collection bags for their return to patients and for such sample's preservation;
ffff. monitoring of subcellular particles derived from pulse flow apheresis system, continuous flow ultracentrifuge rotor system with attached affinity chromatography columns with flow cytometer (FCM), atomic force microscope (AFM), nanoparticle tracking analysis (NTA) system, with disc centrifuge nanoparticle analysis (DCNA) system and large-scale targeted proteomics assay resource;
gggg. monitoring of subcellular particles derived from pulse flow apheresis system, continuous flow ultracentrifuge rotor system with attached affinity chromatography columns and array centrifuge with a series of array centrifuge rotors with flow cytometer (FCM), atomic force microscope (AFM), nanoparticle tracking analysis (NTA) system, with disc centrifuge nanoparticle analysis (DCNA) system and large-scale targeted proteomics assay resource;
hhhh. biochemical and molecular analysis of sucrose density fractions with a photometer and sucrose density gradient fraction collector;
iiii. biochemical and molecular analysis of sucrose density gradient fraction's passed through chromatography columns;
jjjj. collecting blood, red blood cells, leucocytes, lymphocytes, platelets and plasma into collection bags attachable to pulse flow apheresis system;
kkkk collecting continuous flow ultracentrifuge, chromatography columns and array centrifuge processed plasma into sterile collection bags for transfusion back to patient or for preservation and future use;
llll. distribution of purified patient specific subcellular particles, proteomics and genomics for research;
mmmm. distribution of purified patient specific subcellular particles, proteomics and genomics collected with pulse flow apheresis system, continuous flow ultracentrifugation and array centrifuge centrifugation for inter-laboratory analysis and standardization.
